(19) United States
(12) Patent Application Publication
(10) Pub. No.: US 2008/0146940 A1
(43) Pub. Date: Jun. 19, 2008

(54) External and Internal Ultrasound Imaging System

(75) Inventors: David A. Jenkins, Flanders, NJ (US); Charles Bryan Byrd, Medford, NJ (US); Praveen Dala-Krishna, Sicklerville, NJ (US)

Correspondence Address: HANSEN HUANG TECHNOLOGY LAW GROUP, LLP, 1725 EYE STREET, NW, SUITE 300, WASHINGTON, DC, 20006, US

(73) Assignee: EP MEDSYSTEMS, INC., West Berlin, NJ (US)

(21) Appl. No.: 11/610,866

(22) Filed: Dec. 14, 2006

(51) Int. Cl. A61B 8/00

(52) U.S. Cl. 600/463

(57) ABSTRACT

An ultrasound imaging system includes a catheter-based ultrasound transducer, an external ultrasound transducer and a compact ultrasound unit including an isolation circuit, an ultrasound signal generator, and a beam former within a single unit. The ultrasound unit may include circuitry and software to enable simultaneous imaging by both transducers. The isolation circuit may limit unintended, leakage current from the ultrasound system through the transducer array of the ultrasound system.

### FIELD OF THE INVENTION

The present invention is a medical diagnostic system and method, and more particularly is directed to an ultrasound catheter system having transducer arrays positioned both internal and external to a patient's body.

### BACKGROUND OF THE INVENTION

Recent advancements in miniaturization of ultrasound technology has enabled the commercialization of catheters including phased array ultrasound imaging transducers small enough to be positioned within a patient's body via intravenous cannulation. By imaging vessels and organs, including the heart, from the inside, such miniature ultrasound transducers have enabled physicians to obtain diagnostic images available by no other means.

While ultrasound imaging catheter systems have proven to be invaluable diagnostic tools, the associated cabling and equipment present difficulties for clinicians. The cabling from the ultrasound system to the catheter's proximal connector is heavy, stiff, and limited in length. The length of the cabling required to reach from the ultrasound system to the patient can act as an antenna introducing electronic noise induced from stray electromagnetic radiation in the examination room. The large cart which normally contains the ultrasound system takes up space in the operating room or catheterization lab, and may be difficult to position next to the patient. These limitations have made it impractical to perform ultrasound imaging of the heart using transducers positioned internally (e.g., with intracardiac ultrasound catheters) and externally (e.g., a transducer positioned on the chest). As a result, clinicians have had limited ability to combine and correlate ultrasound images from these two viewing perspectives in order better diagnose and treat heart conditions.

### SUMMARY OF THE INVENTION

The various embodiments provide compact, portable ultrasound systems which can operate two or more ultrasound transducers simultaneously, and in certain embodiments include both an intrabody, percutaneous ultrasound imaging probe and an external ultrasound imaging probe suitable for simultaneous examination of an organ from internal and external perspectives.

An embodiment of the present invention includes a compact, integrated ultrasound pulse generation, beam forming, and electrical isolation unit (“ultrasound unit”) with connectors for connecting to two or more ultrasound transducer arrays and an image display unit. Any ultrasound transducer arrays may be connected to the unit. In certain embodiments, one ultrasound transducer array is adapted for intrabody use on a patient (e.g., an intracardiac ultrasound imaging catheter) and the other ultrasound transducer array is adapted for placement on the skin of the patient. The compact ultrasound unit is adapted to be placed on or near the patient to enable the external ultrasound transducer to be positioned on the patient (e.g., the chest) while maintaining suitable sonic contact with the body. In an embodiment, an external transducer array is incorporated into the housing of the compact ultrasound unit.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain features of the invention.

FIG. 1A is a block diagram of an embodiment of the present invention.

FIG. 1B is a block diagram of another embodiment.

FIG. 2 is an illustration of an intra-cardiac catheter located in the right ventricular cavity.

FIG. 3 is a diagram of a catheter-based ultrasound transducer array with a temperature sensor.

FIG. 4 is a diagram of an external ultrasound transducer array.

FIG. 5 is a schematic of an isolation and temperature monitoring circuit according to an embodiment.

FIG. 6 is a block diagram of an ultrasound unit according to an embodiment.

FIG. 7 is a block diagram of an image processing computer of an embodiment.

FIG. 8 is a sample display of an ultrasound image from a cardiac ultrasound transducer.

FIG. 9 is block diagram of another embodiment.

FIG. 10 is an illustration of an embodiment in use on a patient.

FIG. 11 is an illustration of a combined ultrasound unit and external ultrasound transducer assembly according to an embodiment.

FIG. 12 is an illustration of simultaneous ultrasound imaging of a heart using an ultrasound transducer positioned within the heart and an external ultrasound transducer position on the outside of the body.

FIG. 13 is an illustration of an example connector for an embodiment.

### DETAILED DESCRIPTION

Various embodiments of the present invention will be described in detail with reference to the accompanying drawings. Wherever possible, the same reference numbers will be used throughout the drawings to refer to the same or like parts.

As used herein, the terms “about” or “approximately” for any numerical values or ranges indicate suitable dimensional tolerances that allow the part or collection of components to function for their intended purposes as described herein. Also, as used herein, the terms “body” “patient”, “host”, and “subject” refer to any human or animal subject and are not intended to limit the systems or methods to human use. Further, embodiments of the invention will be described for use with an intracardiac ultrasound transducer array catheter. However, the embodiments may be applicable to any medical ultrasound transducer.

The various embodiments provide ultrasound systems and methods that enable simultaneous ultrasound imaging by two or more transducers. Simultaneous imaging of an organ, such as the heart, from both inside and outside the body may offer a number of diagnostic and therapeutic advantages. Limitations on external ultrasound imaging of organs are well known and include interference from bones (e.g., ribs) and connective tissue, and attenuation of ultrasound in blood and tissue which limits the depth within the body at which images can be obtained. While intrabody ultrasound imaging enables unique and close-up views of an organ without interference from bones, the limited viewing depth of intrabody ultrasound may limit the ability to view the entire organ, particularly tissues on the outside of the organ. Thus, viewing the organ from the outside looking in and the inside looking out may reveal details not available by either method alone. Performing such dual perspective viewing simultaneously (instead of in two separate procedures) enables viewing the same structures under the same conditions.

For moving organs, such as the heart, such simultaneous ultrasound imaging from the outside and inside offer a way to correlate ultrasound images obtained from the two perspectives. For example, if an ultrasound scan of the heart is being performed to diagnose and treat a heart arrhythmia, (e.g., atrial or ventricular fibrillation, or ventricular dyssynchrony), it is important to image the heart during the episode of arrhythmia. Since arrhythmia events may vary in initiation, duration, nature and termination, it is also important to image the same episode of arrhythmia in order to properly diagnose and treat the condition. This best can be accomplished by simultaneous ultrasound imaging of the organ from the two perspectives.

Clinicians may also manipulate multiple transducer arrays to better image organs, such as moving the transducers sequentially in order to confirm details imaged or enhance images. For example, one transducer may be moved while both transducers are imaging in order to image with a first transducer areas that are within a sonic shadow of a second transducer. As another example, when one transducer images a feature of interest, the other transducer(s) may be moved or manipulated to image the feature from another perspective or at another frequency.

Combining multiple transducer and internal and external ultrasound imaging capabilities into a single system enables new examination protocols. For example, images from two two-dimensional (2-D) transducers arrays imaging the same region can be used to produce three-dimensional (3-D) images. Since these 3-D images are obtained from two or more simultaneous images, the result may be a 3-D movie (sometimes referred to herein as a 4-D imaging). This capability can enable a clinician to view the 3-D shape and movement of the imaged organ. In cardiac imaging applications, such capability may facilitate understanding the disease conditions affecting the heart and locate sites for applying therapies (e.g., implanting pacing electrodes). This capability may enable detection of cardiac tissue anatomy shaping and movement, electrical energy focus, and timing throughout the cardiac cycle. Furthermore, such 4-D imaging sequences may be fused or otherwise linked to ECG signal information received from ECG sensors and pacemaker pacing signals so the clinician can observe the dynamic 3-D shape response of the heart to natural and therapy pacing signals.

Despite the potential advantages of simultaneous ultrasound imaging from two (or more) transducers, such systems have not been practicable due to several mechanical and operational difficulties posed by using multiple ultrasound transducers. The various embodiments overcome these mechanical and operational difficulties.

The equipment and cabling historically associated with ultrasound imaging present ergonomic challenges for clinicians. For example, the cabling from a conventional ultrasound machine to an ultrasound transducer array is heavy, stiff, and limited in length. The stiffness of the connector has made it impractical to position an ultrasound transducer on the skin of a patient without a clinician maintaining the probe in sonic connection with the skin. The flexure of the connector tends to lift a portion of the transducer away from the skin. Thus, in procedures where there is insufficient room for a clinician to hold an external transducer on the patient or a fixed scanning location is desired, the use of an external ultrasound has been impractical. In particular, intracardiac ultrasound imaging procedures have not been able to include simultaneous external ultrasound imaging since the size and cabling of the ultrasounds systems made placement of the external transducer on the patient impractical. Also, the large cart which normally contains the ultrasound system beam former and processor occupies valued space in the operating room or catheterization lab, and positioning the cart close to the patient may restrict where or how staff or equipment may be stationed. Additionally, the long cable required to reach from the ultrasound system to the patient can be a source of electronic noise as it may act as an antenna that can pick up stray electromagnetic radiation, such as from utility power and nearby computers, power supplies, displays, pulse generators, and other equipment. Further, maintaining sterility of the cable and external transducer connecting to a non-sterile system is a persistent concern in catheterization procedures, such as intracardiac echocardiography procedures.

Beyond the mechanical difficulties of ensuring an external ultrasound transducer remains in proper sonic connection with the body, simultaneous ultrasound imaging presents a number of signal generation and processing challenges which must be overcome to prevent signals of the two (or more) sensors from interfering with each other. Ultrasound pulses emitted by one transducer will be received by the other transducer. Such direct transmission ultrasound will arrive at the receiving transducer with much higher amplitude (i.e., louder) than ultrasound reflected from the tissues and blood being imaged. Thus, two ultrasound transducers operating simultaneously can overwhelm or “jam” each other without some mechanism or method for deconflicting the signals of the two (or more) transducers. Additionally, ultrasound from emissions of one transducer will be received by the second transducer via multiple transmission paths through the body as ultrasound reflects off of various structures. Such multi-path signals will be received at different times (due to the different path lengths) and from a variety of angles resulting in noise that will interfere with imaging if not removed or otherwise accommodated.

To overcome these and other challenges that have stood in the way of simultaneous multi-transducer ultrasound imaging, particularly simultaneous outside-in and inside-out ultrasound imaging, the various embodiments employ a compact ultrasound unit and one or more of a variety of techniques for deconflicting ultrasound signals emitted by the transducers.

Using a compact ultrasound unit resolves many of the cabling and physical layout problems described above by enabling the transducer to be placed close to or integrated with the ultrasound unit. A compact ultrasound unit is disclosed in U.S. patent application Ser. No. (TBD) entitled “integrated Beam Former and Isolation For An Ultrasound Probe” filed contemporaneously herewith, the entire contents of which are hereby incorporated by reference. Various embodiments of the compact ultrasound unit separate the ultrasound generation and beam-forming circuitry from the image display portion of the system, reduce the size of the ultrasound generation and beam-forming circuitry, and integrate the circuitry with electrical isolation circuitry. The result of these innovations is a small, reusable ultrasound unit which houses at least the signal generation, beam-forming, and isolation circuitry in a package can be located adjacent to or on the patient. Unlike a conventional ultrasound cart, the small ultrasound unit can be sterilized or placed into a sterile enclosure (e.g., a sterile plastic bag) and placed on or immediately next to the patient. As a result, the cable from the ultrasound unit to the transducers, particularly the external transducer, can remain relatively short and provided with particular shapes or configurations to facilitate maintaining proper sonic connection between the transducer and the patient. Also, the short length of the transducer cable means that it may be economically feasible to configure it as a sterile, single-use commodity which may make it easier to use in procedures also involving intrabody catheterization, such as intracardiac echocardiography. A lighter weight, inexpensive, disposable cable or a wireless communication interface can connect the ultrasound unit to an image display unit. The image display unit of the system may then be positioned at any convenient location, such as on the bed next to the patient or even on the patient.

As described herein, a number of ultrasound signal deconflicting and coordinating techniques are implemented in various embodiments to enable simultaneous ultrasound imaging of the same organ by two or more transducers. By way of example but not by way of limitation, such techniques described in greater detail herein include: alternating ultrasound pulses; synchronizing ultrasound pulses; employing different ultrasound frequencies in each transducer; including identifying codes in each ultrasound pulse; signal processing techniques; and combinations of two or more of these techniques. Such techniques may be implemented within the compact ultrasound unit in circuitry, software and combinations of circuitry and software as described herein.

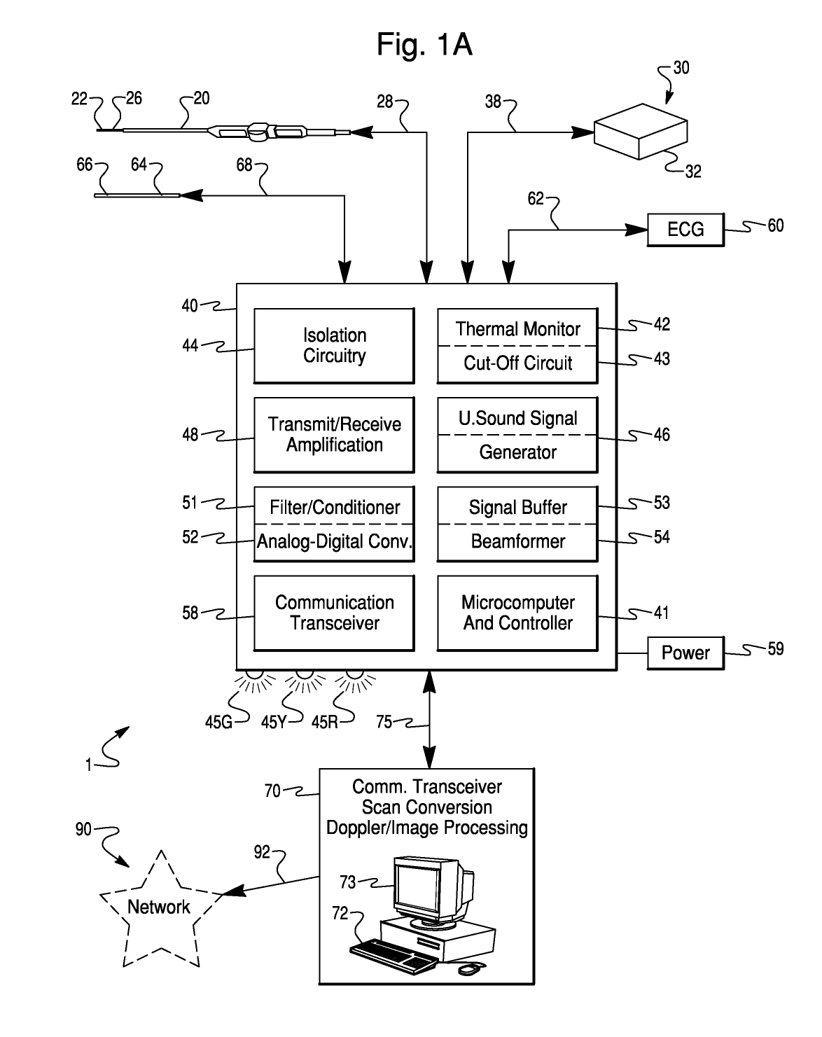

The block diagram in FIG. 1A illustrates main elements of an example embodiment. The embodiment includes an ultrasound transducer array 22 carried by or positioned on a catheter 20 coupled to an ultrasound unit 40 by a signal cable 28, and an ultrasound transducer array 32 on an external transducer assembly 30 coupled to the ultrasound unit 40 by a signal cable 38. While the embodiment illustrated in FIG. 1A includes a catheter-based transducer and an external transducer, this is for example purposes only and is intended to limit the invention to only two transducers or to an external transducer. The ultrasound unit 40 can be connected to a display, such as a display unit 70, by a wired data interface 75 or a wireless data interface 76 shown in FIG. 1B.

A signal cable 28 delivers ultrasound signals from ultrasound unit 40 to each of the transducers in the array 22. Typically, the signal cable 28 will include at least one wire per transducer, and in an embodiment, includes a coaxial cable connected to each transducer in the array 22. In an alternative embodiment, the signal cable 28 includes fewer wires than transducers and a multiplexer circuit (not shown) configured to enable signals to and from the plurality of transducers over the wires. Typically, the signal cable 28 includes an electrical connection plug (e.g., a standard connector) on the proximal end. Providing a plug connector on the end of the cable 28 allows completion of the many electrical connections between the cable conductors and the ultrasound unit 40 since the connection by pressing the plug into a complementary connector in the housing 100 of the ultrasound unit 40.

Similarly, a signal cable 38 delivers ultrasound signals from ultrasound unit 40 to each of the transducers in the external transducer array 32. Typically, the signal cable 38 will include at least one wire per transducer, and in an embodiment, includes a coaxial cable connected to each transducer in the array 32. In an alternative embodiment, the signal cable 38 includes fewer wires than transducers and a multiplexer circuit (not shown) configured to enable signals to and from the plurality of transducers over the wires. Typically, the signal cable 38 includes an electrical connection plug (e.g., a standard connector) on the proximal end. Providing a plug connector on the end of the cable 38 allows completion of the many electrical connections between the cable conductors and the ultrasound unit 40 since the connection by pressing the plug into a complementary connector in the housing 100 of the ultrasound unit 40.

The transducers in the arrays 22, 32 convert the electrical signals from the ultrasound unit 40 into sound waves, which propagate into a portion of a patient's anatomy, such as the heart. The same transducer arrays 22, 32 also receive ultrasound echoes reflected from anatomic structures and transform the received sound into electrical signals (e.g., by means of the piezoelectric effect). These electrical signals are conducted via cables 28, 32 back to the ultrasound unit 40.

While FIG. 1a shows a single ultrasound catheter 20 and external ultrasound assembly 30, the ultrasound unit 40 may include connectors and internal circuits for connecting more internal and external ultrasound sensors. Such additional ultrasound sensors may be included by providing the ultrasound unit 40 with additional circuits as described herein or including multiplexing circuits so that the same circuits can provide signals to and process signals from such additional ultrasound sensors.

The ultrasound unit 40 may include a housing or chassis with exterior connectors for connecting cables to other elements of the embodiment. The ultrasound unit 40 may contain optical and electronic circuitry implementing some or all of the elements described in the following paragraphs. The component elements and interconnecting circuitry of the ultrasound unit 40 may include one or more large scale integrated circuits such as VLSI, ASIC, and FPGA chips mounted on one or more circuit boards which are coupled to the connectors.

A signal generator 46 generates electrical signals of ultrasonic frequencies to be provided to the ultrasound transducer arrays 22, 32. The signal generator 46 can be configured to produce signals of particular wave forms, frequencies and amplitudes as desired for imaging tissue. The signal generator 46 may also be configured to generate signals with the necessary phase lag to enable the transducer array to generate a focused and steerable sound beam as well known in the art of imaging ultrasound phased array transducers. Alternatively, phase lag may be added by another circuit, such as a beam former circuit 54.

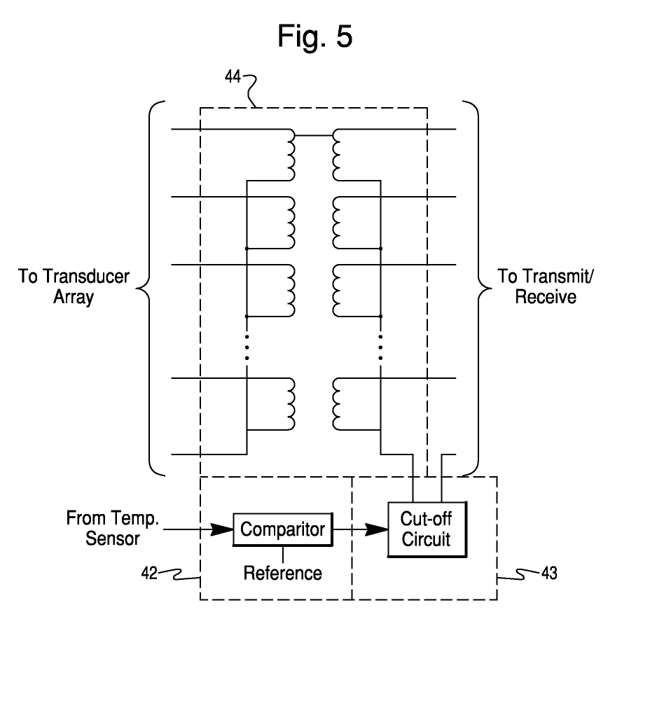

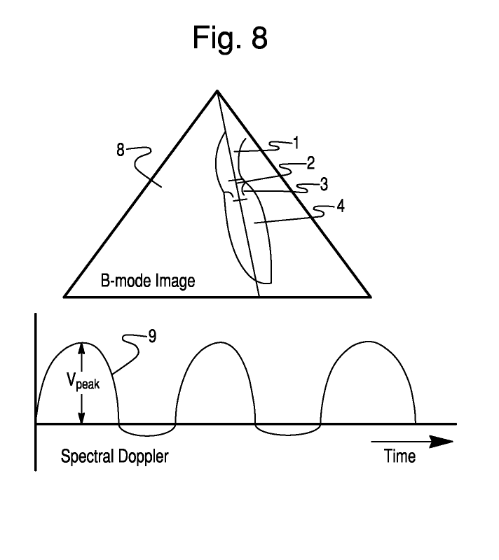

In an embodiment, a single signal generator 46 generates electrical signals for both ultrasound transducer arrays 22, 32. A multiplexer circuit, such as transmit/receive multiplexer circuits 48 illustrated in FIGS. 5 and 8, may be coupled to the signal generator in this embodiment to alternately provide the generated electrical signals to the appropriate one of the two ultrasound transducer arrays 22, 32. In an alternative embodiment, two or more signal generators 46 are provided in the ultrasound unit 40 (e.g., block 46 includes two or more signal generator circuits), with each signal generator supplying electrical signals to a corresponding one of the ultrasound transducer arrays 22 or 32. In either configuration, the signal generator 46 may be configured to generate ultrasound signals that include one or more deconflicting techniques as mentioned above and described more fully herein. For example, a single signal generator 46 may alternate between generating a set of electrical signals directed to the internal ultrasound transducer array 22 and generating a set of electrical signals directed to the external ultrasound transducer array 32, so that the two sensor alternatively transmit/receive ultrasound pulses. As another example, the signal generator 46 may generate electrical signals of two (or more) different frequencies, with signals with one frequency directed to the internal ultrasound transducer array 22 and signals of the other frequency directed to the external ultrasound transducer array 32.

In addition to the optional functionality described above, a transmit/receive multiplexer circuits 48 illustrated in FIGS. 5 and 8 can be included to direct the signals generated by the generator 46 to isolation circuitry 44 and to separate out echo signals returned from isolation circuitry 44 from the generated signals and direct those signals to signal processing circuits. Further, the transmit/receive multiplexer circuits 48 may be configured to direct signals received from isolation circuitry 44 to different processing circuits depending upon whether the received signals were received from the internal or external ultrasound sensors 20, 30. Thus, the transmit/receive multiplexer circuits 48 may play a role in deconflicting ultrasound signals from the two (or more) ultrasound sensors as described more fully herein.

Isolation circuitry 44 isolates unintended, potentially unsafe electrical currents and voltages from the transducer array 22 and, optionally, 32. Examples of suitable isolation circuits are described in U.S. patent application Ser. No. 10/997,898 “Method And Apparatus For Isolating A Catheter Interface”, published as U.S. patent Publication No. 2005/0124898 to Borovsky et al. filed on Nov. 29, 2004, the entire contents of which are hereby incorporated by reference. An example of such safety methods and systems is embodied in the ViewMate® catheter ultrasound system from EP MedSystems, Inc. of West Berlin, N.J. Since the external ultrasound assembly 30 is intended for use outside the body, less stringent isolation design criteria may be imposed for connections to this sensor. Alternatively, the same isolation circuits may be used for all connected ultrasound sensors to provide enhanced patient safety, to enable use of standard connectors for all ultrasound cables or to enable employing two internal ultrasound sensors. The isolation circuitry 44 or other form of electrical isolation is required for the intracardiac transducer array 22 due to safety concerns with stray or induced electrical fields introduced into the heart. However, the same degree of electrical isolation may not need to be provided for an external transducer 32 since the body is more resistant to electrical fields applied to the skin. Thus, no isolation circuitry may be provided for the external transducer connection, or if isolation circuitry is provided, it may be designed to less stringent requirements for leakage voltage, current and resistance.

A thermal monitoring circuit 42 and a cut-off circuit 43 may be included to mitigate possible risks to the patient that can result from excessive local heating by internal ultrasound transducers. For example, the thermal monitoring circuit 42 may be connected to a temperature sensor (not shown), such as a thermoresistor (“thermistor”) positioned on the catheter near the transducer array 22. The thermal monitoring circuit 42 is preferably configured to determine from signals received from the temperature sensor when tissue temperatures in the vicinity of the transducers exceeds a safe threshold value and to trigger a safety action when the threshold is exceeded. The safety action may be the output of a cut-off signal to a cut-off circuit 43 which is configured to shut off the signal generation, disconnect transmit circuits from the transmission cable 28, or otherwise discontinue the transmission of ultrasound pulses to the transducer array 22 in response to a cut-off signal. Examples of suitable temperature sensors, thermal monitor circuits and cut off circuits are provided in U.S. patent application Ser. No. 10/998,039 entitled “Safety Systems And Methods For Ensuring Safe Use Of Intra-Cardiac Ultrasound Catheters” published as U.S. patent Publication No. 2005/0124899 to Byrd et al. filed on Nov. 29, 2004, the entire contents of which are hereby incorporated by reference. The thermal monitor circuit 42 may be provided only for connections to subcutaneous transducer arrays since the body is more resistant to ultrasound heating applied to the skin.

In an embodiment, the thermal monitoring circuit 42 is configured to monitor intra-cardiac temperatures as sensed by a thermistor on the catheter, and to transmit to the display unit 70 a temperature value that may be displayed on a monitor. The thermal monitoring circuit 42 in such an embodiment may calculate the intracardiac temperature value and transmit this value as digital data to the display unit 70. The thermal monitoring circuit 42 may also be configured to transmit a warning when the measured intracardiac temperature exceeds a threshold, such as a temperature that is elevated but still safely below a level at which tissue damage may occur. Such a warning would inform clinicians of a potentially hazardous condition to permit them to take actions to reduce heating, such as adjusting ultrasound power or duty cycle parameters, in order to avoid damaging tissue and automatic shutoff by the cut-off circuit 43. Such a warning may be transmitted to the display unit 70 as digital data for display on the monitor.

In another embodiment, a display, such as colored light emitting diode (LED) indicators (45G, 45Y, 45R) on the ultrasound unit 40 are provided to indicate temperature information, in the alternative or in addition to displays on to the display unit 70. For example, in such an embodiment three LEDs may be provided, such as a green LED 45G to indicate a safe detected intracardiac temperature, a yellow LED 45Y to indicate an elevated but marginally safe intracardiac temperature, and a red LED 45R to indicate an unsafe or near unsafe intracardiac temperature. In such an embodiment, the thermal monitoring circuit 42 includes circuits configured to light the appropriate colored LED based upon the measured intracardiac temperature. This configuration may be accomplished by the thermal monitoring circuit 42 testing the sensed temperature against two threshold values, wherein the first threshold corresponds to elevated but still safe intracardiac temperatures and the second threshold corresponds to unsafe or near unsafe intracardiac temperatures. Thus, the thermal monitoring circuit can be configured (e.g., with digital switches) to power (i.e., direct a voltage to) the green light emitting diode in response to the temperature input signal indicating a sensed temperature less than the first threshold, power the yellow light emitting diode in response to the temperature input signal indicating a sensed temperature greater than the first threshold but less that the second threshold, and power the red light emitting diode in response to the temperature input signal indicating a sensed temperature greater than the second threshold.

The filter and conditioning circuit 51 can be included in the ultrasound unit 40 to reject spurious signals that may be induced in or through cables 28 and 32.

An analog-to-digital converter (ADC) 52 can be included in the ultrasound unit 40 to frequently sample and convert the ultrasound signals from analog electrical levels to discrete digital numeric values. The analog-to-digital converter 52 may include a single ADC circuit that performs the conversion for all signals received from both transducer arrays 22, 32, or may be configured with two or more ADC circuits with each performing the conversion of signals received from one of the transducer arrays 22 or 32.

A signal buffer 53 can be included to store at least a portion of the echo signals, which are returned from the transducer arrays 22, 32 and which may be processed by other elements of the ultrasound unit 40. In an embodiment, a signal buffer 53 is included to store the echo signals as digital data in a random-access semiconductor memory (RAM). Storage of echo signal data may be provided in separate tables, with associated metadata tags or stored in addresses according to a preset sequence so that data from the two (or more) transducer arrays can be recovered or transmitted separately. Alternatively, the signal buffer 53 may include separate RAM chips with the buffer configured to store echo signal data from one of the transducer arrays 22 or 32 in a corresponding one RAM.

Beam former 54 circuits may be included to process signals sent to and received from the transducer arrays 22, 32 to enable phased-array ultrasound imaging. The beam former 54 may receive ultrasound signals from the signal generator 46 and introduce phase lags for each transducer element so that when the signals are applied to the transducer elements a narrow beam of sound emanates from the arrays. Also, the beam former 54 may receive signals from the transducer arrays 22, 32 and process the ultrasound echo signal data to calculate the amplitude and direction of the ultrasound echoes returned to the transducer arrays 22, 32 from each of many specific angles and distances. The beam former 54 may also determine the frequency or Doppler frequency shift of the signal returned form each of selected angles and distances from the transducer arrays 22, 32. As with other components within the ultrasound unit 40, the beam former 54 may include a single beam former circuit that processes signals to/from each ultrasound transducer 22 or 32, or two or more beam former circuits that each process signals from a corresponding one (or more) of the two transducer arrays 22 or 32. For example, if signals from the two ultrasound transducers 20, 30 are distinguished by time phasing pulses (i.e., alternating pulses so the transducers do not transmit simultaneously), then the beam former 54 may include only a single beam forming circuit since the signals from the two transducers are separated in time. Alternatively, for example, if signals from the two ultrasound transducers 20, 30 are distinguished by frequency (i.e., they emit different ultrasound frequency pulses), the beam former 54 may include two (or more) beam former circuits with each filtered or configured (or both) to receive and process one of the two (or more) frequencies.

A communications transceiver 58 may be included to prepare ultrasound data for transmission out of ultrasound unit 40, typically in digital form. The communication transceiver 58 may also receive data and commands from outside the ultrasound unit 40 and convert such signals to a form usable by the ultrasound unit 40. Data transmission may by any high speed (e.g., gigabit per second) data link, such as Ethernet.

In an embodiment, the communications transceiver 58 may include data encoding or compression capability, such as a microprocessor programmed with data encoding or compression software, so that the ultrasound data can be transmitted in a compressed format. By transmitting data in a compressed format, lower bandwidth communication links (e.g., cable or wireless data link) can be used to transmit data, or more data can be transmitted over a standard cable or wireless data link. Additionally, the control unit 41 or other modules may be configured to filter out data that need not be transmitted, such as signals or data pixels containing little or no data (i.e., pixels where little or no echoes were received), so such data need not be transmitted. In yet a further embodiment, the communications transceiver 58 may include temporary storage capability (e.g., random access memory) and be configured to manage the transmission of data at a maximum data rate consistent with the communication link even when data provided to the transceiver exceeds the maximum data rate. Suitable circuitry and software for encoding, compressing, buffering and filtering data and managing data transmission are well known in the communications arts.

The ultrasound unit may include a control unit 41 which may be a microcontroller, a microprocessor, a microcomputer, or other controller circuitry (such as programmable firmware or a programmable gate array). The control unit 41 may be configured to coordinate the activity and functionality of the various elements included in the ultrasound unit 40. In an embodiment, two or more control units 41 may be included, with one dedicated to controlling the operation and processing of the ultrasound signals to/from to a respective one of the two transducer assemblies 20 or 30. In another embodiment, the control unit 41 may include a processor dedicated to one of the two (or more) transducer assemblies 20, 30, and a separate processor configured to coordinate the dedicated processors (e.g., to coordinate pulse timing, configurations or self testing) and other global functions of the ultrasound unit 40.

In an embodiment associated with cardiac imaging, the ultrasound unit 40 may also include electrical connections for receiving signals from electrocardiogram (ECG) electrodes and for passing such signals on to an external ECG unit 60 which may be connected to the ultrasound unit 40 through a communications interface 62. The communications interface 62 may be any wired or wireless interface. In an embodiment, the ECG electrodes can be an intracardiac ECG catheter 64 which includes one or more electrodes 66 near a distal end for sensing electrical activity in the heart. Electrical signals sensed by the electrodes 66 can be conveyed to the ultrasound unit 40 by means of an extension of the catheter 64 or a connecting cable 68. In various embodiments, the ECG catheter 64 is connected to the isolation circuitry 44 which isolates the patient from stray or fault voltage from the external ECG equipment 60. In an embodiment, signals sent by the ECG 60 through the interface 62 can be recorded or used to synchronize received ultrasound image data with the heartbeat of the patient. For example, a sequence of images may be associated with a sequence of ECG readings revealing the phases of the cardiac cycle, or images may be captured only at a specified phase of the cardiac cycle.

In an embodiment in which the ultrasound unit 40 may be packaged within a single housing (see FIGS. 5 and 8) or chassis, the whole unit 40 may be fabricated of components which can withstand a sterilization method. Sterilization methods include subjecting the unit 40 to gas, liquids, heat (dry or steam), radiation, or other known methods. Alternatively, or in addition, the unit 40 may be enclosed in an externally sterile enclosure, such as a plastic bag, with provision for connecting cables through the plastic bag to the unit 40. For example, the connectors may be designed so that the pins of the connector of external electrical cables (such as cables 28 and 38) are designed to puncture the externally sterile plastic bag locally when mating with the corresponding connector of the unit 40 inside the plastic bag.

The relationship, function, and interaction of the elements which may be contained in the ultrasound unit 40 of an embodiment will be described further with reference to FIGS. 5 and 8.

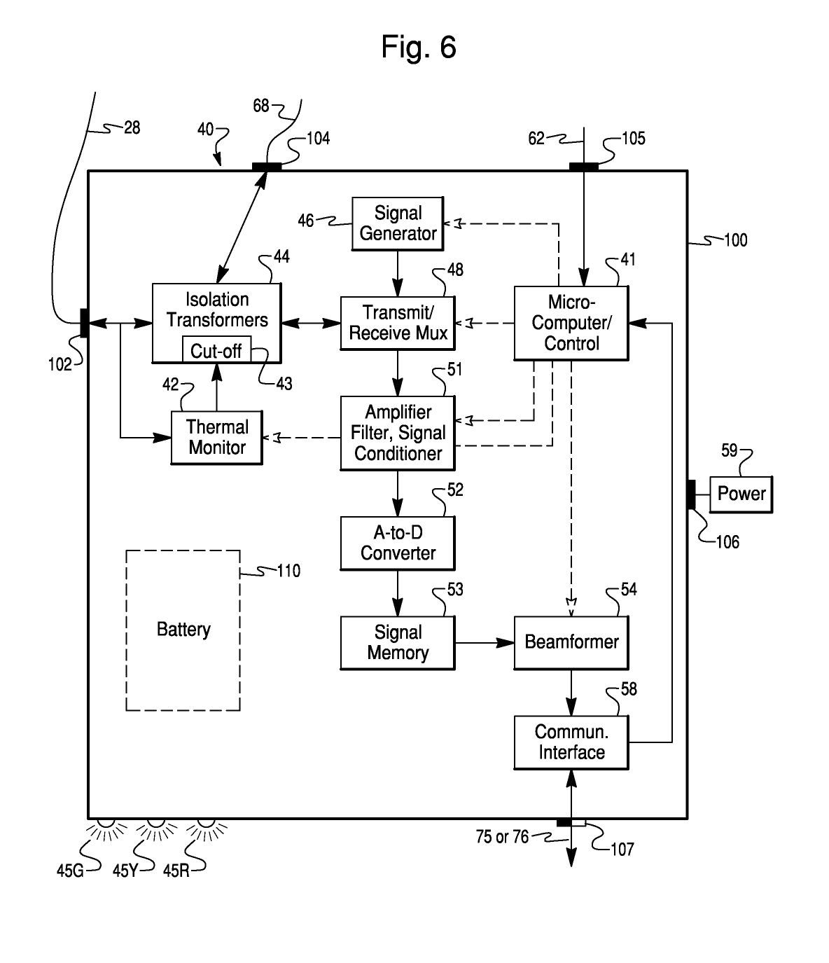

In FIGS. 1A and 1B, the image display unit 70 may be a computer, such as a laptop, which can be configured to perform more sophisticated image processing than is provided by the ultrasound unit 40. Such an embodiment will be described later (with reference to FIG. 6). In an alternative embodiment (which will be discussed later with reference to FIG. 8) image display computer may include a user input device 72 and a video monitor 73.

Optionally, there may be two (or more) separate communication interfaces 75: one interface for the ultrasound image data communicated to the display unit 70, and a second interface (not shown in FIG. 1A) for communicating configuration parameters and commands from the display unit 70 to the control unit 41. These two interfaces 75 may employ the same type of communication hardware and protocol standard, or one type of communication hardware and protocol standard for sending ultrasound data and a different type of communication hardware and protocol standard for receiving commands and configuration instructions from a user input device 72.

In the embodiment illustrated in FIG. 1A, pixel-based, polar-coordinate oriented ultrasonic image data can be serialized and transmitted to the image display unit 70 over the data communication interface 75. Such data may also be tagged to indicate which of the two (or more) transducers from which it was received, or transmitted in groups or alternating sequences so that the display unit 70 can process, store and display the data from each of the transducers appropriately. The data interface 75 may be any one or more of several standard high-speed (e.g., gigabit/second) serial or parallel data communication protocols and hardware embodiments. Example embodiments of the serial communication interface 75 include Ethernet, Universal Serial Bus (USB 2.0), FireWire (IEEE-1394), RS-232, or any other existing or future high-speed wired communication interface. As with the communications transceiver 58, the data interface 75 may include circuitry and software for encoding, compressing, buffering and filtering data, as well as managing data transmission to enable the reliable transmission of a large amount of image data through communication links of limited bandwidth. Both the ultrasound unit 40 and the image display unit 70 can contain appropriate corresponding hardware and software communication drivers, data compression and encoders, buffer memory, and data filter circuits and/or software, which are readily available commercially, such as standard off-the-shelf integrated circuits or plug-in circuit cards and well known communication algorithms.

In an alternative embodiment, the data interface 75 may be an optical cable, such as one or more fiber optic cables. This embodiment is illustrated in FIG. 1A, with the communication transceiver 58 being an optical data link transceiver. Fiber optic data links well known in the art may be used in this embodiment.

In some embodiments, the communications transceiver 58 and/or the data interface 75 are configured to recognize the particular type of communication link connected to the ultrasound unit 40, and to adapt the communication protocols, data encoding and data transmission rates to match the connected link. Such embodiments may also include software programmed in the control unit 41 that enables it to supervise the ultrasound unit 40 consistent with the capabilities and requirements of the connected data link. In such embodiments, the ultrasound unit 40 may include a number of different connection ports for various communication links, such as two or more of a USB port, a FireWire port, a serial data port, a parallel data port, a telephone band modem and RJ-11 port, a WiFi wireless data link and a BlueTooth wireless data link, for example. Circuitry and/or software operating in the communication modules or microprocessor 41 can be configured to sense when a particular one of the various accommodated communication links is connected, such as by sensing an electrical or radio frequency signal received by the link. Recognizing that a particular link is connected, the communications transceiver 58 and/or the data interface 75 and/or microprocessor 41 can implement the protocol, data encoding and data transmission rate that corresponds to that link. Circuits and methods for recognizing connected data links and adjusting communications protocols accordingly are well known in the digital communication arts. Such embodiments provide flexibility of use, allowing users to connect displays and processors using available or convenient cables or communication links.

Functionality within the ultrasound unit 40 can be managed and timed by a control unit 41, which may be a programmed microcontroller, microprocessor, or microcomputer, equivalent firmware, functionality included within an ASIC chip, or discrete electronic circuitry, all of which are encompassed by description references to “control unit” herein. The control unit 41 can respond to configuration parameters and commands sent from the image display unit 70 over communication interface 75. Examples of such configuration parameters and commands include the frequency of the generated ultrasound signals, the mode of operation (continuous or pulsed), depth of imaging, angular width of the active image area, amplifier gain, filter frequencies, details about the transducer arrays 22, 32 (number and arrangement of transducers), and so forth.

The hardware layout and software programming needed to implement the design and programming of the ultrasound unit 40 are typical and well known to electrical and software engineers skilled in this art. Similarly the algorithms programmed into display unit 70 are known to software engineers skilled in mathematics, computer graphics, and graphical-interface operating systems.

The image display unit 70 can perform any number of several functions. The display unit 70 can process and display the image data provided by the ultrasound unit 40 on connected monitor 73 or other display, such as a large plasma screen display (not shown) coupled to the display unit 70. The display unit 70 can transmit configuration parameters and control commands to the ultrasound unit 40, where the configuration parameters and commands may be supplied by the operator of the system 1 by means of interactive inputs from a pointing device (mouse or joystick) and keyboard attached to or part of the display unit 70. For example, the operator may inform the display unit 70 about the type of imaging catheter 20, which the display unit 70 may further translate into operational details about the transducer arrays 22, 32 included in the imaging catheter 20.

In some embodiments, the image display unit 70 can convert the ultrasound data generated by the beam-former 54 (which may be relative to a transducer-centered polar coordinate system) into one or more images relative to another set of coordinates, such as a rectangular coordinate system. Additionally, image data from both transducer arrays 22, 32 may be combined into an integrated display. Such processing may not be necessary in the display unit 70, if the conversion was already preformed in the ultrasound unit 40. Techniques for converting image data from one coordinate system into another are well-known in the field of mathematics and computer graphics.

The display unit 70 may display the rectangular image data as an image on a standard video monitor 73 or within a graphics window managed by the operating system (such as Microsoft Windows XP) of the display unit 70. In addition, the display unit 70 can display textual data for the operator on the monitor 73, including, for example, information about the patient, the configuration parameter values in use by the ultrasound unit 40, and so forth. In an embodiment, the display unit 70 may provide a function that allows measuring the distance between two points on the image, as interactively selected by the operator.

To analyze and display an indication of the motion—and specifically the velocity—of locations in the image corresponding to tissue and fluid movement (i.e., blood), further Doppler frequency distribution analysis can be performed and translated into a readily understandable graphical representation. Doppler frequency analysis is well-known in the field of ultrasound medical imaging and described in more detail in the patent applications incorporated by reference herein. Fourier analysis may be used, for example, to determine the frequency distribution information and the average Doppler frequency shift for each of all points or selected points in the ultrasound image, and from which to compute the individual velocities of those points.

The display unit 70 can generate an image in which the Doppler frequency shift information communicated by the ultrasound unit 40 for each point or pixel is represented by a color hue. Since Doppler shift provides information on the speed and direction of movement of fluids and structures with respect to the transducer, various color hues can used in the display to correspond to the velocity and direction of motion. For example, red may be used to represent the maximum velocity in one direction, blue to represent the maximum velocity in the opposite direction, and colors between red and blue on the color spectrum to represent velocities in between. In another embodiment, such colors can be superimposed on the B-mode image, which is otherwise rendered as a gray-scale image wherein the brightness of each pixel depends on the amplitude of the returned ultrasound echo from the anatomical location corresponding to the pixel. Other modes of display are well known in the art, such as a plot of the distribution of Doppler frequency shifts at the points along a line in the image, and M-mode in which the movement along narrow lines is displayed.

Optionally, a connection interface 92 may connect the display unit 70 to a clinic or hospital information infrastructure 90 or the Internet. A hospital information infrastructure 90 will typically include a network of attached workstations, graphical displays, database and file servers, and the like. The optional interface 92 typically can be an Ethernet cable, a wireless WiFi interface (e.g., IEEE 802.11), or any other high-speed communications physical layer and protocol. For example, an interface 92 can allow the display unit 70 to access information from the Internet, a database, or a hospital network infrastructure 90. The display unit 70 may also transmit information outward via the interface 92, such as to store the ultrasound data on a network server or display the ultrasound images elsewhere than the display unit 70.

FIG. 1B illustrates another embodiment which electrically isolates the ultrasound unit 40 from the image display unit 70. The embodiment illustrated in FIG. 1B uses a wireless data communication interface 76 to convey ultrasound image data to the image display unit 70. This embodiment may have safety advantages, because it electrically isolates the ultrasound unit 40 from the image display unit 70 and eliminates a data cable which can pick up and conduct stray electrical fields and electronic noise. By removing an electrical conduction path for high voltage or leakage currents, namely the wires or cable of the interface 75, between the ultrasound unit 40 and the display unit 70, this embodiment provides further patient protection from potential internal or external electrical faults and eliminates a source of electronic noise. Not only does this embodiment provide added protection from faults within display unit 70, but it can provide protection from lightening or power surges which may occur with an embodiment such as a laptop or desk-top personal computer plugged into a normal AC utility power outlet. Without sufficient isolation circuitry, lighting or surges can send a high voltage spike through the power supply of the display unit 70 and into the rest of the system 1 through any available conductive pathway.

Commercially available wireless communications systems can be used for the wireless interface 76. For example, the wireless interface 76 may be an infrared communication interface (such as the IRDA standard), a radio-based communication interface (such as the Bluetooth, ZigBee, or 802.11 WiFi or 802.15.4 standards), or both (for example, infrared in one direction and radio in the other direction). To decrease the data processing that must be done in the ultrasound unit 40, a high speed data transmission system may be used to provide partially processed ultrasound data to the image display unit 70 for further processing. The data transmission may have a data rate of 1 megabit per second or more.

Common to the embodiments illustrated in FIGS. 1A and 1B is a power supply 59 coupled to the ultrasound unit 40. Electrical power is used both to power the processors and circuits in the ultrasound unit 40 and to provide energy for the electrical pulses which drive the transducer arrays 22, 32. Power may be provided through the data cable 75 (as in the case of a USB cable embodiment), via a separate power cable connected to the display unit 70, via a separate power supply (such as a transformer connected to an AC power source), or via a self contained power source (such as a battery). For an embodiment in which the image display unit 70 does not supply power to the ultrasound unit 40, such as where the image data is communicated via a wireless data link as illustrated in FIG. 1B, the ultrasound unit 40 can be powered by a separate power source 59. Non-limiting examples of suitable separate power sources include a rechargeable battery pack, a disposable battery pack, a power supply connected to public utility power lines through an isolation transformer, a power supply engineered for safety compliance isolation from public utility power, a solar cell, a fuel cell, a charged high-capacity storage capacitor, combinations of two or more of such power sources (e.g., a rechargeable battery and a solar cell), and any other source of electrical power which may become known in the art.

A self-contained power source, such as a battery or solar cell, can provide inherent safety advantages over conventional power sources. This is because a self-contained power source 59 can be used to further isolate the patient from stray and fault currents since there need not be a power cable connected to the beam former unit. This removes the power source (such as hospital main AC power) and the power cord as sources of power spikes and fault currents. Such isolation may be further enhanced by forming the housing or chassis of the ultrasound unit 40 from non-conductive material and encasing the self-contained power source within the housing or chassis. This configuration effectively presents no return path or common ground between the power source and the patient. That is, even if there is an electrical potential difference between the ultrasound unit 40 and the patient, little current can flow between them. Further, with a self-contained power source, there is very little chance that a patient will receive high voltage from lightening or from a utility power outlet through a failed component, faulty design, or a power surge. Still further, a typical self-contained power supply 59 can supply only limited current at limited voltage, further reducing the likelihood of excessive leakage current. Also, using a self-contained power supply 59 can further reduce cables and conductors which can pick up stray electromagnetic radiation and become a source of electronic noise in the system.

In another embodiment, the power supply 59 may be power conductors parallel to or contained in the communication cable 75 of FIG. 1A, over which the image display unit 70 provides power to the ultrasound unit 40. The display unit 70 can supply power from its own supply of power such as, for example, if the interface 75 connecting the display unit 70 and the ultrasound unit 40 is embodied by a standard IEEE-1394 (Firewire) cable or a USB cable, both of which contain direct current power conductors.

If an embodiment does use an electrically conductive cable as part of the communication interface 75, then in lieu of other measures, the data communication interface 75 can include additional circuits for isolating the ultrasound unit 40 from any source of excessive leakage current or high voltage from or through the display unit 70 whether or not power is supplied over the interface 75. National and international safety organizations specify leakage current and breakdown voltage standards for various medical applications; for example, a maximum leakage current of 20 microamperes or a breakdown voltage of at least 5000 volts. By providing electrical isolation circuitry 44 within the ultrasound unit 40, greater protection for the patient can be provided against high power, power surges, and system overloads, as well as providing greater protection against or filtering of signal artifacts, signal jitter, and signal crosstalk.

Further patient electrical isolation is provided in embodiments utilizing a fiber optic data cable 75 between the ultrasound unit 40 and the display unit 70. In an alternative embodiment further isolation may be provided by including an optical isolator module somewhere along each conductor of the interface 75. An example of an optical isolator module includes a light-emitting diode optically coupled to a photo detector, and configured so that electrical signals entering the module are converted into light signals and converted back into electrical signals within the module, thereby conveying the data across an electrically isolating space. In embodiments where the data interface 75 is an optical fiber with suitable optical-electrical converters at each end of the interface 75, the optical fiber cable can be constructed to prevent or minimize electrical conduction, such as fabricating the covering from non-conducting plastics, thereby providing better electrical isolation. Optical isolation may preclude supplying electrical power through the data interface 75, so an alternative power supply 59 for the ultrasound unit 40 according to an embodiment described herein may be employed. The use of optical fiber cable or an optical isolation can also help to reduce electronic noise introduced into the ultrasound unit 40 from stray electromagnetic radiation.

In the various embodiments, the ultrasound unit 40 lies between the patient on side and power sources and external processors/displays on the other. As such, different levels of isolation may be provided by separate isolation circuitry 44 within the ultrasound unit 40 as appropriate to particular connections. For example, by providing greater electrical isolation on connections to power and/or external processor/displays, such isolation may protect the circuitry of the ultrasound unit 40 as well as the patient from external voltage spikes and fault currents. As another example, isolation circuitry on the patient side of the ultrasound unit 40 circuitry, may reduce hardware and software complications and increase integration efficiency.

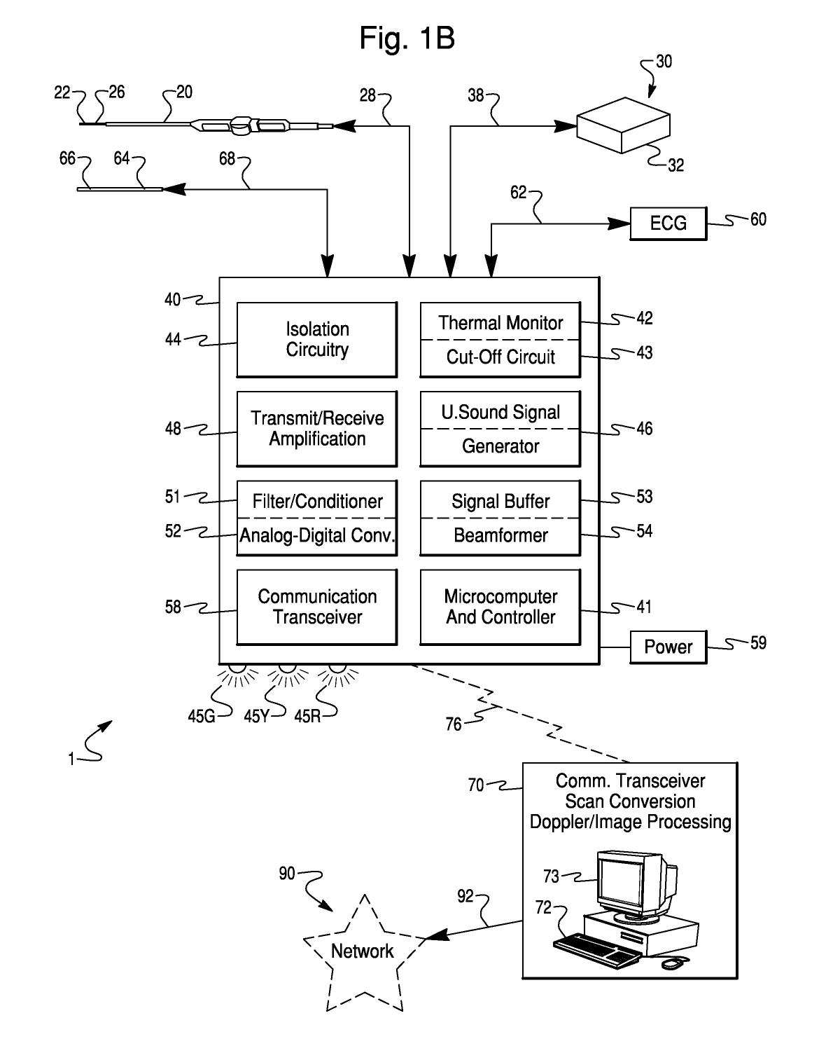

FIG. 2 depicts a simplified cross section of a human heart 12 with an ultrasonic imaging catheter 20 positioned in the right ventricle 14. The catheter 20 includes an ultrasound transducer array 22, which may image at least a portion of the heart 12. For example, the imaging view 26 afforded by the transducer array 22 may allow imaging the left ventricle 13, the ventricular walls 15, 16, 17, and other coronary structures. Usage of an embodiment may include positioning the array 22 at other locations and at other orientations within the heart (such as the right atrium), within a vein, within an artery, or within some other anatomical lumen. Insertion of the catheter 20 into a circulatory system vessel or other anatomical cavity through use of a percutaneous cannula is well known in the medical arts.

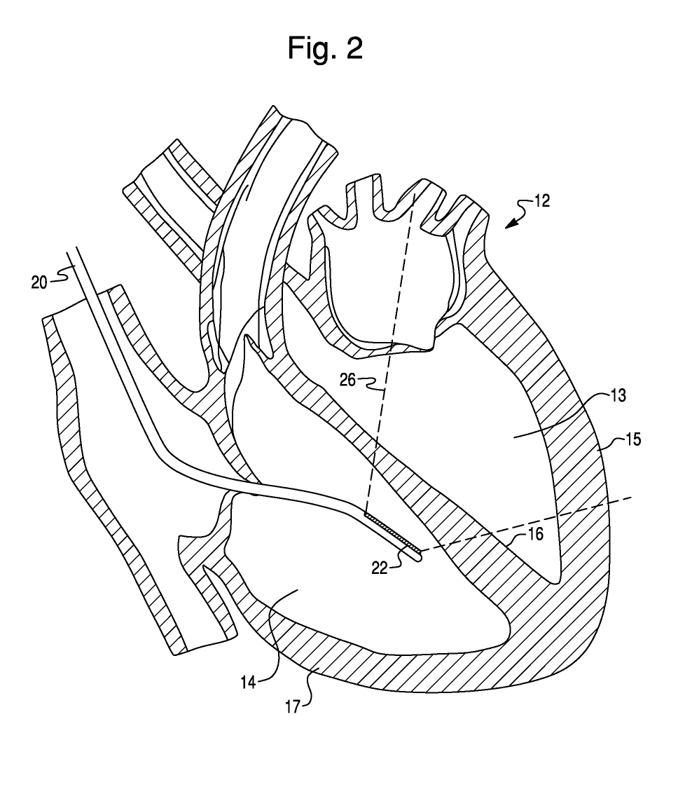

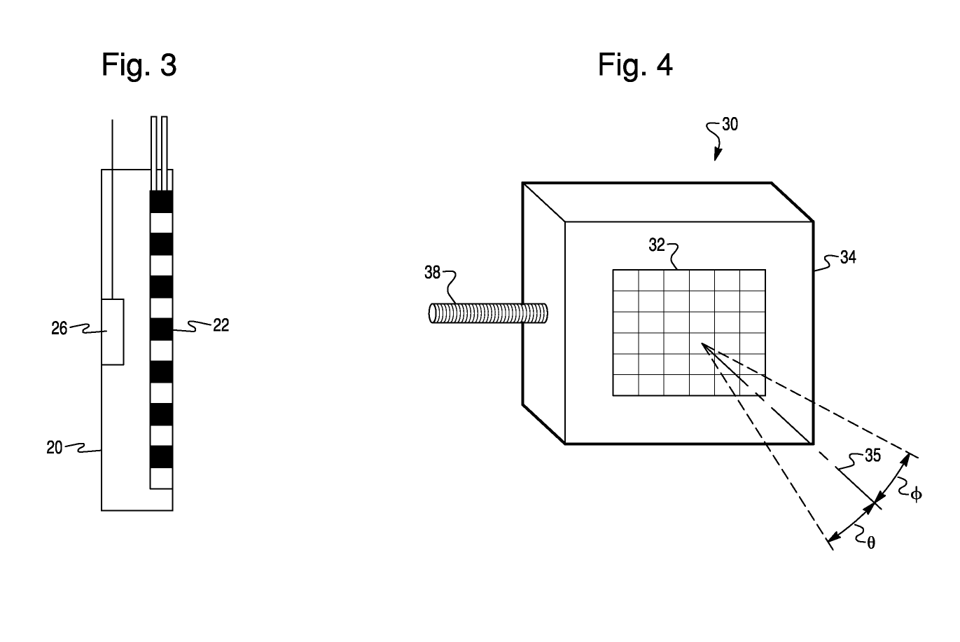

FIG. 3 is a close-up example of an embodiment of a portion of a catheter 20, carrying an ultrasound transducer array 22. The array 22 may be located near the distal end of the catheter 20, but may be located elsewhere within the to catheter 20. Also shown in FIG. 3 is a temperature sensor 26 for connection to the thermal sensing circuit 42 and cut-off circuit 43 shown in FIGS. 1A and 1B.

Examples of phased array ultrasound imaging catheters used in performing intracardiac echocardiography and methods of using such devices in cardiac diagnosis are disclosed in the following published U.S. patent applications—each of which is incorporated herein by reference in their entirety.

2004/0127798 to Dala-Krishna et al.;

2005/0228290 to Borovsky et al.; and

2005/0245822 to Dala-Krishna et al.

### Commercially available ultrasound catheters are available from EP MedSystems, Inc. of West Berlin, N.J.

It should be noted that the present invention is not limited to the specific catheter assembly disclosed in the applications cited above, because the invention is applicable to various catheters and instruments designed for intravascular and intracardiac echocardiography and for other physiological uses involving an ultrasound beam former and interfaces with medical instruments and external display equipment.

FIG. 3 illustrates an external ultrasound transducer array 30 according to an embodiment. A number of commercially available ultrasound imaging transducers may be used with the ultrasound unit 40 according to various embodiments. Such a transducer assembly 30 will typically include a transducer array 32 positioned on the surface or behind a sonic window of a housing 34 connected to a cable 38 for connecting the transducer assembly 30 to the ultrasound unit 40. An external ultrasound transducer array 32 may be a two-dimensional or three-dimensional imaging phased array. A two-dimensional imaging phased array includes a linear array of transducer elements which can be pulsed by the ultrasound unit 40 so as to produce an ultrasound beam that can be directed through a viewing angle within a plane (the plane including the linear array and a line perpendicular to the emitting face of the transducers). Thus, a two-dimensional imaging array can generate image data in the two dimensions of angle θ with respect to the array and distance from the array. A three-dimensional imaging array may include two (or more) linear arrays of transducers oriented at an angle to one another, such as perpendicular to one another. For example, a three-dimensional imaging array may be a square or rectangular array of transducer elements, which can be viewed as a number of linear arrays positioned side-by-side. The transducer elements can be selectively pulsed with a phase relationship (i.e., with different delays or with different pulse phases) so as to produce an ultrasound beam that can be directed at any angle to the face of the array. Thus, a three-dimensional imaging array can generate data in the three dimensions of two angles with respect to a normal 35 to the array face (angle θ and declination φ) and distance from the array.

For all medical imaging technologies, patient safety is of paramount concern. For imaging technologies involving intrabody probes (e.g., ultrasound imaging catheters, electrophysiology (EP) catheters, ablation catheters, etc.), particular attention is paid to protecting the patient from unintended electrical currents and power emissions within the patient's body. For example, testing has shown that leakage currents of sufficient strength can cause muscle stimulation, which may be detrimental to the patient undergoing intrabody imaging. As such, industry approved electrical safety standards (e.g., for isolation, grounding, and leakage current) have been established for medical devices, such as national standards set by the Association for Advancement of Medical Instrumentation, limiting leakage currents from intracardiac probes to less than 50 microamperes.

In typical catheter based probes, electrical shielding or insulation is provided by way of a robust catheter body to satisfy the industry approved electrical safety standards. Shielding alone, however, may be unsatisfactory for some implementations, as substantial shielding increases the thickness of the catheter body. Induced currents may also arise from the catheters acting as an antenna picking up energy radiated by electronic equipment present in a typical electrophysiology lab. In some instances, the shielding may be inadvertently damaged and, thus, not provide adequate protection. Thus, methods and devices that enable intracardiac medical devices to meet or exceed the federally mandated electrical safety standards are highly desirable.

Published research has revealed that the human heart is more vulnerable to small currents when the currents are introduced within the heart itself, such as by percutaneous catheters. In Cardiovascular Collapse Caused by Electrocariographcally Silent 60 Hz Intracardiac Leakage Current, by C. Swerdlow et. al., the authors reported that leakage currents as low as 20 microamps may induce cardiovascular collapse when applied within the heart. Accordingly, percutaneous catheters might require greater electrical isolation than specified in more general standards in order to assure patient safety.

Such small leakage currents can readily arise, for example, from imperfect electrical insulation, condensation in circuits, faulty electronic components, ambient radio waves, and induction from surrounding circuits and magnetic fields. Further, safety standards require minimum (“creepage”) distances (such as 5 millimeters) between certain conductors to isolate a patient from possible high voltage discharges resulting from unlikely but possible component failures.

FIG. 5 shows an embodiment of the isolation circuitry 44 and thermal monitor circuit 42. In this example embodiment, electrical isolation is accomplished by a transformer circuit for each transducer in the catheter transducer array 22. Transformers do not conduct direct current and small air-core transformers can be used that will not readily pass low frequency alternating current (such as the 50 to 60 hertz of standard utility power outlets). Thus, electrical isolation is provided by presenting high impedance to unintended direct current and to lower frequency alternating currents. Further, if insulated adequately, the transformers will not conduct D.C. voltages below some very high, specified break-down voltage.

Another example of a safety concern for intrabody ultrasound systems is the sustained power of ultrasound radiated from the transducers, specifically for the higher power employed by color Doppler imaging, wherein the power may locally heat tissue above a safe body temperature. Although the ultrasound generation electronics may indirectly limit the amount of heat an ultrasonic catheter can theoretically induce in tissue at a given power level, direct monitoring of the actual temperature at the transducer array and surrounding tissue is much safer and avoids assumptions about how effectively specific tissues can dissipate the heat. Therefore, a need exists for a safety means either to warn the operator or to curtail the applied power automatically, whenever the measured temperature exceeds some pre-determined limit. An example of a standard safe temperature limit, as established by FDA in the United States, is 43 degrees Celsius, although the exact limit may depend on the specific environment and use of the catheter.

Besides the ultrasound transducer array 22, the catheter 20 may optionally further include an electronic temperature sensor 26, such as a thermocouple or thermistor, as shown in FIG. 3. The purpose of the temperature sensor 26 is to measure the increase in temperature resulting from the injection of high-power ultrasound into living tissue. Because the sound energy is most concentrated near the ultrasound transducer array 22, the temperature sensor 26 is best located very close to the array 22, such as on the catheter 20 containing the array 22. Temperatures above a proscribed level (such as 43 degrees Celsius) can permanently damage tissue and must be avoided. Therefore, the temperature sensor 26, together with the thermal monitor circuit 42 of FIG. 5, can be calibrated to detect temperatures above the proscribed level. When the temperature exceeds the proscribed temperature, the cut-off circuit inhibits or at least reduces the generation of the ultrasound signals generated by the signal generator 46. This is accomplished in FIG. 5 by interrupting the common conductor to which all the transformers are connected.

FIG. 5 illustrates an example embodiment of the thermal monitor circuit 42 that includes a thermal comparator circuit 42 coupled to a cut-off circuit 43. In this embodiment, signals received from a thermocouple or thermistor 26 positioned near the catheter transducer array 22 can be compared in a comparator circuit to a reference threshold value corresponding to a maximum safe temperature. If the sensed temperature signal exceeds the reference threshold, indicating temperatures in the vicinity of the catheter transducer array 22 exceed a safe temperature, the comparator circuit can generate a cut-off signal that is provided to the cut-off circuit 43. In the circuit embodiment illustrated in FIG. 5, a common lead is provided to all transformers, particularly on the transmit/receive side (i.e., the portion of isolation circuit connected to the ultrasound unit 40). This circuit permits the cut-off circuit 43 to be a simple switch that opens to disconnect the common lead on the transmit/receive side of the isolation circuit in response to a cut-off signal received from the temperature comparator circuit 42. As mentioned above, the thermal monitoring circuit 42 need not be coupled to an external ultrasound transducer.

Another example embodiment of the thermal monitor circuit 42 includes a plurality of gate circuits configured to gate the individual leads passing signals to and from each of the transducer elements in the catheter transducer array 22. So long as the temperature measured by the thermistor 26 remains below a safe level (e.g., not more than 43° C.), the gate circuits remain enabled allowing signals to pass to/from the transducer elements. However, should the temperature measured by the thermistor 26 reach or exceed an unsafe level, the thermal monitoring circuit 42 disables the gate circuits, automatically shutting off the catheter transducer array 22. In another example embodiment, the thermal monitor circuit 42 is configured to disable transmission of ultrasound signals from the ultrasound unit 40 by disabling the transmit circuitry by signaling the signal generator 46 through a trigger mechanism, such as a hardware interrupt signal. Other thermal monitor circuit embodiments include circuits that disable an array of multiplexers or transmit channel amplifiers that may be used in the ultrasound unit 40 for generating, controlling, distributing, conditioning and/or transmitting ultrasound pulses to the catheter transducer array 22. The various example embodiments of the thermal monitor and cut-off circuits 42, 43 as well as other suitable circuits, perform the safety function of discontinuing transmission of ultrasound signals from the signal generator 46 through the isolation circuit 44 upon receiving a cut-off signal or sensing an unsafe temperature in the vicinity of the catheter transducer array 22.

FIG. 6 illustrates example connections among components of the ultrasound unit 40. The embodiment illustrated in FIG. 6 is not intended to specify the only possible configuration of components and their interconnections but serves as an example of an enabling implementation.

Referring to FIG. 6, the signal generator 46 generates electrical signals of ultrasonic frequencies, such as in the range of about 1 megahertz to 10 megahertz, as are commonly used for ultrasound imaging. The signals may be continuous or may be intermittent pulses. The electrical signals may pass through a transmit/receive multiplexer 48, isolation circuits 44, and a cable 28 to reach the catheter transducer array 22 and/or a cable 38 to reach the external transducer array 32. The electrical signals, which reach the transducer arrays 22, 32, cause the transducers to produce ultrasound signals in the same frequency range generated by the generator 46.

The transmit/receive multiplexer 48 directs the signals generated by the generator 46 through the isolation circuitry 44, which serves to limit unwanted electrical currents and voltages passing into the cables 28, 38.

Signals which transit the isolation circuitry 44 pass into the signal cables 28, 38, which delivers the signals to each of the transducers in the arrays 22, 32. The cables 28, 38 may include at least one wire per transducer in the array. The transducers in the arrays 22, 32 convert the electrical signals into sound waves, which propagate into a portion of a patient's anatomy, such as the heart. As shown in FIG. 6, the same cable 28, 38 (or a separate cable, not shown) may conduct the return ultrasound signals back to the isolation circuitry 44 of the ultrasound unit 40.

An embodiment of the isolation circuitry 44 is described above with respect to FIG. 5. In an embodiment of the ultrasound unit 40, the isolation circuitry may be connected to the cable 28 and, optionally cable 38, with no other active circuitry in between. This arrangement may prevent a possible, compromised path for leakage current caused by other circuitry. An embodiment employs each conductor of cables 28, 38 both to conduct the generated electrical signal to each transducer of arrays 28, 38 and to conduct the returning echo signal from the transducer. Thus, both the generated signal and the return signal may pass through the same isolation circuit (transformer, bi-directional optical isolator, or other electrical isolation component). In an embodiment there may be at least one isolation circuit for each transducer of arrays 28, 38. In an embodiment, the isolation circuits 44 are coupled to the catheter cable 28 (i.e., to the internal transducer array 22) but not to the external ultrasound assembly cable 38 since there are less stringent limitations on allowable leakage currents for external sensors. As mentioned above, isolation circuitry provided for external ultrasound transducers need not be designed to the same electrical tolerances on leakage current or voltage as for subcutaneous catheter transducer arrays.

Multiplexer 48 may be necessary where the generated signals from the generator 46 and the received echo signals both pass through the same isolation circuitry 44 and the same wires of cables 28, 38. Specifically, the transmit/receive multiplexer 48 may be used to separate out the received echo signals from the generated electrical signals from the generator 46. This may be accomplished by connecting the transducer arrays 22, 32 to the receiving amplifier circuits between generated ultrasound pulses, so the only signals allowed to pass are the received ultrasound echo signals. Alternatively, the electrical pulses of the transmitted pulses may be subtracted from all signals received from the isolation circuitry to yield the received ultrasound echo signals. The multiplexer 48 can direct the received ultrasound echo signals to conditioning circuitry 51 to filter and condition the signals as necessary. The multiplexer 48 may also differentially direct signals corresponding to the catheter transducer array 22 to one set of filter and conditioning circuits and signals corresponding to the external transducer array 32 to a different set of filter and conditioning circuits. The signals received from the transducer arrays 22, 32 can also be amplified, such as within the multiplexer 48, within the conditioning circuitry 51, or by a separate amplifier or amplifiers. At some point, the amplified and filtered echo signals can be digitized by an analog-to-digital converter (ADC) and can be temporarily stored in a signal memory buffer 53, such as random access semiconductor memory (RAM).

For an embodiment which employs separate transducers and conductors for the generated signals and for the received echo signals, separate transformers or mono-directional optical isolators may be used in the isolation circuitry 44. In such an embodiment, the transmit/receive multiplexer 48 may not be included.

The filter and conditioning circuitry 51 may be provided to reject signal frequencies outside a certain range, such as rejecting spurious signals induced in the cables 28, 32, for example. In an embodiment in which the internal transducer arrays 22 and external transducer arrays 32 emit pulses at different frequencies, the filter and conditioning circuit 51 may also separate echo signals from the two arrays by frequency filtering (e.g., using notch filters tuned to one of the ultrasound frequencies). The echo signals may be amplified as part of this circuitry (before, during, and/or after filtering), as part of the transmit/receive multiplexer 48 or elsewhere in the ultrasound unit 40. Other signal processing may be performed to enhance the desirable properties of the signal returned from the transducer arrays 22, 32. For example, the signal conditioning circuitry 51 may reject the spurious signals from internal reflections within the catheter.

The analog-to-digital converter (ADC) 52 can be included to frequently sample the received ultrasound signals, which are received as analog electrical levels, and convert them to discrete digital numeric values. This conversion may be performed before, during, or following the action of the filter and conditioning circuitry 51. The signal may be in either analog or digital form or both during parts of the amplification, filtering, conditioning, and storage of the signal. In other words, the filter and conditioning circuitry 51 may apply well-known analog filtering techniques to the signals before digitization, may apply well-known digital filtering methods to the digitized signals, or do both. Techniques for such digital and analog signal processing are well known. There may be an ADC per transducer, or fewer ADCs may used with each being time-multiplexed among multiple transducers. Conversion of signals to streams of digital numbers is a convenience based on the current availability and economy of digital processing, but suitable analog circuits can be used in part or all of the ultrasound unit 40.

Because contemporary electronics routinely store signals in digital form, the embodiment of FIG. 5 may digitize the return echo signals prior to storage of some portion of the return signals in memory 53, which may be semiconductor random-access memory (RAM). Methods of storing an analog signal, such as in time delay circuits, may be used instead of or in addition to digital storage. A portion of the memory 53 may store the signal from each transducer of arrays 22, 32. In an embodiment, the memory 53 may be partitioned to store signals from the catheter transducer array 22 in one portion of the memory 53 and store signals from the external transducer array 32 in another portion of the memory 53. In another embodiment, the memory 53 includes two or more memory chips with one memory chip configured and used to store signals from the catheter transducer array 22 and another memory chip configured and used to store signals from the external transducer array 32.

The beam-former 54 processes the returned echo signal data, some or all of which may be stored in memory 53. The beam-former 54 may calculate the amplitude of the ultrasound echos at each of many specific angles and distances from the transducer arrays. Techniques for beam-forming (either for transmission or for reception) are well known in the fields of ultrasound imaging and in phased array sonar and radar.

The result of the processing of the data stored in buffer memory 53 by the beam-former 54 typically can be a pixel-based image relative to a polar-coordinate system. The beam-former 54 may be implemented in very large scale integrated (VLSI) semiconductor circuits. The beam-former 54 may employ substantial parallel processing of the ultrasound signals from the transducers of arrays 22, 32. For example, there may be simultaneous computations of more than one beam angle and/or more than one distance along a beam angle. In an embodiment, the beam-former 54 includes a single VLSI circuit that is configured to process ultrasound data from both transducer arrays 22, 32. In another embodiment, the beam-former 54 includes a VLSI circuit configured to process ultrasound data from the catheter transducer array 22 and another VLSI circuit configured to process ultrasound data from the external transducer array 32.

The amplitude, phase and time of arrival of reflected ultrasound pulses at each transducer in the array 22 or 32 can be used by the beam-former 54 to calculate the angle (with respect to the long axis of the catheter transducer array 22 or with respect to the normal 35 to the external transducer array 32) and distance from the array to each echo source. In a 3-dimensional ultrasound transducer array 32, the beam-former 54 may calculate two angles (e.g., longitudinal angle and declination angle) The distance and angle data may then be combined with amplitude (i.e., power of the received echo) to produce (e.g., by processing the data according to an algorithm) a pixel of image data. Alternatively, the beam-former 54 may store or output the processed received ultrasound as data sets comprising data groups of angle, distance and amplitude. In this and like manner, the beam-former 54 can turn the large volume of streaming ultrasound signals into a smaller set of data easily passed over a serial data link 75 for processing or display by a display unit 70. In an embodiment, the beam-former 54 is included in one or more VLSI chips within the ultrasound unit 40.

The beam former 54 may compare the frequency of the generated signals with the frequency spectrum of the returned echo signals. The difference in frequency relates directly to the velocity of tissue or blood toward (higher frequency) or away from (lower frequency) the transducer array due to the Doppler effect. The difference in frequency, i.e., the amount of Doppler frequency shift, is indicative of motion of the tissue (including blood) from which the ultrasound reflected. The frequency shift may be determined by mixing the generated signal and received echo signal and detecting the difference frequency. The conversion of the frequency shift to velocity depends on the speed of sound in the body, which is about 1450 to 1600 meters per second in soft tissue, including blood. The conversion of frequency shift to velocity according to well known algorithms may be performed immediately by the beam former 54, or later, such as by an external image processor at the time the image is displayed. If calculated by the beam-former 54, the velocity data (or Doppler shift) may be outputted as a fourth element of the data set, so that echo sources are identified by angle, distance, amplitude and velocity (or frequency or Doppler shift).

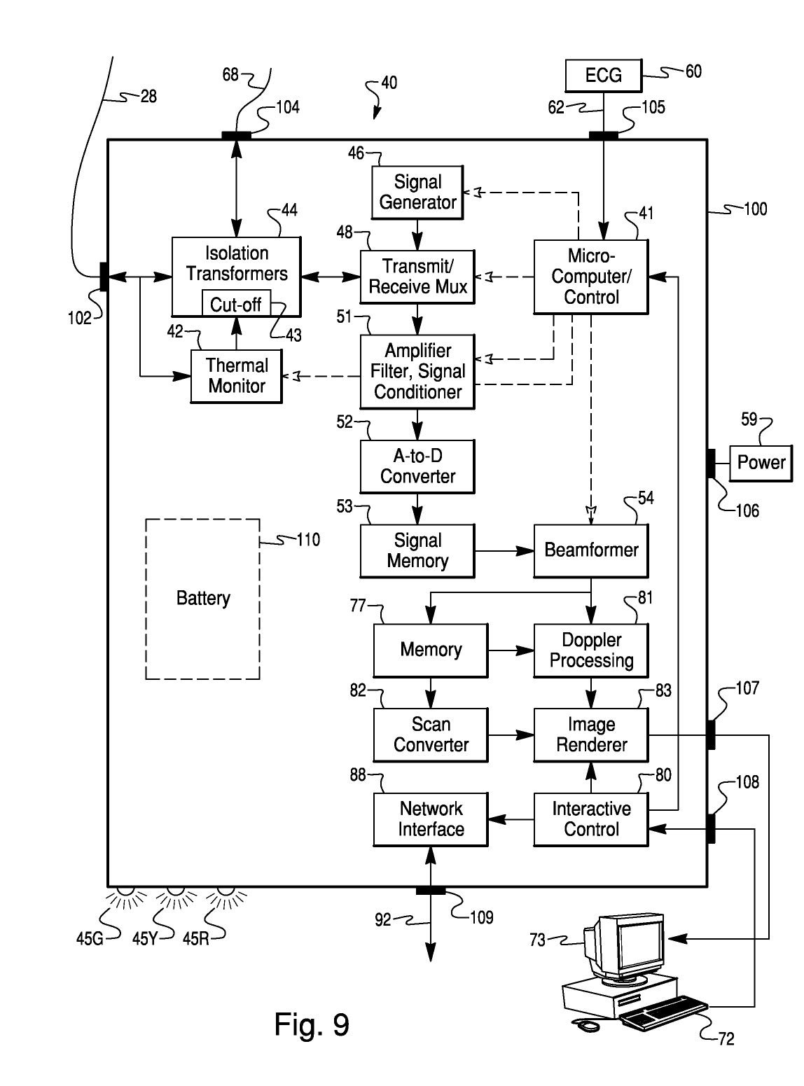

The velocity of echo sources may be computed at each of numerous angles and distances relative to the transducer arrays 22, 32. The computed velocities may be represented visually as a spectrum of colors, for example, as is conventional in the art. The velocity of numerous points in the tissue or blood may be mapped to colors at the corresponding locations in the final image of the tissue. Image display unit 70, as shown in FIG. 1A, may perform this mapping, although it may be performed alternatively within ultrasound unit 40, as shown in FIG. 9.

With reference to FIG. 6, an embodiment of a beam former 54 may directly produce an image in rectangular coordinates. Alternatively, the beam former 54 may directly produce an image in polar coordinates and transform the image into rectangular coordinates. Alternatively, the beam-former 54 may simply produce an image in polar coordinates (i.e., angle and distance coordinates) and allow subsequent image processing to perform the coordinate transformation as needed (such as in image display unit 70). In a further embodiment, the beam-former 54 may combine signals and data from both transducer arrays 22, 32 and generate image data based upon the combined ultrasound information.

As also shown in FIG. 6, the buffer memory 53 may make available the return signal data representing the ultrasound echo waves, and the beam-former 54 may access that data and may calculate the amplitude of the ultrasound echo at each of many specific angles and distances from the transducer array. The result of the processing of the data stored in the buffer memory 53 by the beam-former 54 may be a pixel-based image relative to a polar-coordinate system. In an embodiment as illustrated in FIG. 6, polar-coordinate oriented ultrasound echo data may be serialized and may be transmitted to the image display unit 70 over data interface 75. Alternatively, the beam-former 54 may generate data relative to a rectangular coordinate system and transmit that data to display unit 70 over an interface 75.

As explained with respect to FIG. 1A, a control unit 41, which may be a programmed microcontroller, microprocessor, or microcomputer or functionally equivalent discrete electronics, can be included to coordinate the activity described above within the ultrasound unit 40. In addition, the control unit 41 (or equivalent) may respond to configuration parameters and commands sent from the image display unit 70 (of FIG. 1A) over the communication interface 75 or 76 to the ultrasound unit 40.

In an embodiment, the ultrasound unit 40 may be configured via software or discrete circuits to adaptively cut and separate each frame of ultrasound image data. Such capability may be used to select and transmit frames for which there is useful information (e.g., changes in position of structures) to limit the bandwidth required for transmitting ultrasound images to external displays. In a normal cardiac cycle, portions of the heart are at rest for significant fractions of the cardiac cycle, so numerous images during such intra-contraction periods will contain the same image information. By not transmitting images in which there has been no change since the previous image, the same clinical information may be transmitted at substantially lower data rates. Such processing of image frames may be accomplished by a segmentation module (not shown).

In an embodiment associated with cardiac imaging, an external electrocardiogram (ECG) unit 60 (see FIG. 1A or 1B) may also be connected to the ultrasound unit 40 through an ECG communications interface 62 connected to the ultrasound unit 40 by a connector 105. ECG signals may be sent to the external ECG unit from ECG sensors 66 connected to the ultrasound unit 40. Also, signals from the external ECG unit 60 may be received by the ultrasound unit 40 through interface 62 and used for ultrasound imaging purposes, such as to synchronize ultrasound imaging with the heartbeat of the patient. For example, a sequence of images may be associated with a sequence of phases of a cardiac cycle, or images may be captured only at a specified phase of the cardiac cycle. The ECG communications interface 62 may be any wired or wireless interface. The ECG signal may be monitored by the control unit 41 to enable it to orchestrate the operation and timing of the signal generator 46 in order to image the heart at particular phases of the cardiac cycle.

Referring to FIGS. 1A and 1B, an embodiment may employ one or more ECG sensors 66 integrated in an intravenous catheter 64 that is coupled to the ultrasound unit 40 by a connecting cable 68. In an embodiment, ECG sensors may be included on the catheter 20 which carries the ultrasound transducer array 22, while in other embodiments the ECG catheter 64 is separate from the ultrasound catheter 20. The ultrasound unit 40 may include connectors 104 for receiving (i.e., electrically connecting to) the electrical connection plug from an ECG catheter 64 or connecting to the electrical connection plug from a cable 68. Such connectors 104 may route the ECG signals through the isolation circuitry 44 and then out to an external ECG unit 60 via cable 62. This embodiment allows the ultrasound unit 40 to serve as a universal connector for the ultrasound and ECG instruments used in a typical intracardiac examination employing both ECG and ultrasound sensor. This embodiment reduces the need for multiple cables and connectors, thereby simplifying the procedure.

In an embodiment, signals from the ECG sensor 66 may be used in lieu of, or in addition to, signals from an external ECG unit 60, which may have its own ECG sensor or sensors. The ECG sensor signals can be used to record or control the timing of the ultrasound image acquisition relative to the cardiac cycle instead of or in conjunction with signals from an external ECG unit 60. The signals from an ECG sensor may be included within the data stream output by the ultrasound unit 40.

Whether an ECG signal is acquired from an external ECG unit 60 or an attached ECG sensor 66, the interface 60, 68 (or cable 28 if the ECG sensor is on the ultrasound catheter 20) may be electrically isolated from the ultrasound unit 40 to enhance patient safety and reduce electronic noise in the system. For wired interfaces, the isolation may be accomplished by a transformer isolation circuit 44 or an optical isolator as described herein. Protection against electrical leakage currents and high voltage discharges may be accomplished in an embodiment by using a wireless interface for the ECG interface 62.

In addition to including connectors for receiving the input/output connection plugs for ultrasound catheters and ECG sensors or equipment, some embodiments of the ultrasound unit 40 include connections for additional sensors, such as intracardiac percutaneous leads, subcutaneous leads, reference leads and other electrical leads that may be employed during a procedure. As with ultrasound and ECG connections, such additional lead connections can be coupled to isolation circuitry 44 to provide patient protection. By providing such connections with integrated isolation circuitry, the ultrasound unit 40 can serve as a central interface unit for connecting all sensors employed in a procedure.

Some or all of the electronic circuitry of ultrasound unit 40 may be implemented within one or more large scale integrated circuits such as VLSI, ASIC, or FPGA semiconductor chips, as are well known in the electronics arts. The program instructions running in the control unit 41 may be stored in some form of random access memory (RAM) or read-only memory (ROM) as software or firmware.

Portable ultrasound units, which contain compact signal generation and beam-forming circuitry and which are separate from the display unit 70, are available commercially from Terason, a division of Teratech Corporation (Burlington, Mass.). Some of the details of these portable ultrasound units are described along with associated methods in the following published patent applications—each of which is incorporated herein by reference in its entirety:

2005/0228276 to He et al;

2005/0018540 to Gilbert et al; and

2003/0100833 to He et al.

Also described in the above applications are methods of implementing the beam-forming computations. However, these commercially available instruments lack several elements of the various embodiments.

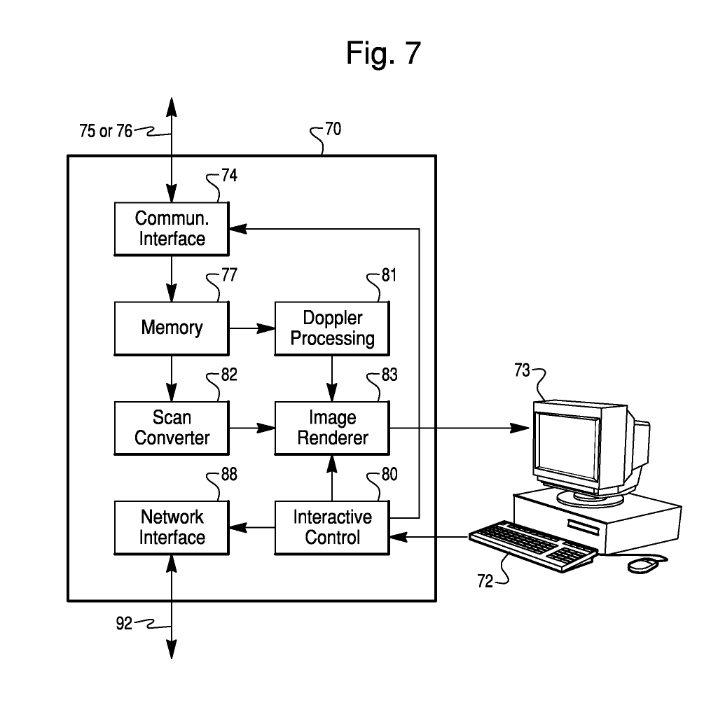

As shown in FIG. 7, the communication interface 74 within the display unit 70 may receive the ultrasound data over the interface 75 or 76 and may temporarily store the data in memory 77 for further processing. The image data at this point may be relative to a polar coordinate system, so scan converter 82 may reformat it into an image relative to a rectangular coordinate system as needed.

Image data from the scan conversion (and Doppler processing, if any) may be processed by an image renderer 83, formatted, and displayed as an image on a video monitor 73. For example, the rendering circuit 83 may generate a gray-scale image (such as a B-mode image) in which the brightness of each pixel is representative of the amplitude of the ultrasound echo from the anatomical point to which the pixel corresponds. Image renderer 83 may also process image data from both the catheter transducer array 22 and external transducer array 32 to generate a combined image. Such a combined image may be two (or more) images, one for each transducer array 22, 32 projected side-by-side, above-and-below or superimposed. Alternatively, a single image may be generated employing data from both transducer arrays 22, 32 to show details or perspective not possible with data from a single transducer array.

Besides responding to operator input of configuration information, the interactive control 80 may also respond to operator input controlling how the images are converted, processed, and rendered on the display 73.

The image display unit 70 may perform other functions as well. For example, the interactive control 80 in the image display unit 70 may transmit configuration parameters and control commands to the ultrasound unit 40, where the configuration parameters and commands may be supplied by the operator are interactive inputs received from a pointing device (mouse, trackball, finger pad, or joystick, for example) and a keypad or keyboard 72 attached to display unit 70.

Optionally, the interactive control 80 of image display unit 70 may forward the image and/or raw data to a network, a network file or database server, the Internet, a display screen, or to a workstation through a communication interface 92.

FIG. 8 illustrates example images that can be displayed by an embodiment. Contained within the field of view 8 of the B-mode image is an image of the walls 5 of a ventricular cavity 4. Also shown in FIG. 8 is a plot 9 of the blood flow velocity as derived from the spectral analysis of the Doppler frequency shifts.

In an embodiment, the image display unit 70 circuitry may be included within the ultrasound unit 40 housing or chassis. This may be accomplished by simply including the image display unit 70 circuitry as another board or VLSI chip within the ultrasound unit 40. Alternatively, the circuitry and functionality of the components of the image display unit 70 may be incorporated in a VLSI chip that also encompasses the beam-former 54 and/or control unit 41 within the ultrasound unit 40. In such an embodiment, the ultrasound unit 40 can output image data as a video signal (e.g., VGA, composite video, conventional television or high-definition video) that can be carried by a cable 75 directly to a display 73 to yield an image on the screen without further processing. In a further embodiment, the ultrasound unit 40 may output image data as a network compatible signal, such as Ethernet or WiFi, that can be directly coupled to a network. In an embodiment including a cable 75, 76 for connecting to a display, a standard connector 107, such as a video connector (e.g., CGA, VGA, EVGA, etc.), Ethernet or USB connector, may be included to permit easy connection to and disconnection from the display and external control hardware.

FIG. 9 is a block diagram of such an embodiment in which most or all of the image processing circuitry is included within the ultrasound unit 40. That is, most or all of the circuitry of FIG. 7 is compactly incorporated into the chassis of the ultrasound unit 40. This is enabled by readily available circuitry equivalent to a personal computer on a small single circuit board or within a single integrated circuit.

In the embodiment illustrated in FIG. 9, the communication interface 58, communication cable 75 (of FIG. 6) and communication interface 74 (of FIG. 7) may be simplified, integrated into a single circuit or essentially eliminated. Further, ultrasound unit 40 may be directly linked to the hospital network and infrastructure 90 (shown in FIGS. 1a, 1b) through a network interface circuit 88 connected to a wired or wireless data link 92 as described above. In an embodiment including a wired data link 92, a standard connector 109, such as a Ethernet, Firewire or USB connector, may be included to permit easy connection to and disconnection from an external network.

A user input device 72 may connected to the ultrasound unit 40 to permit a user to provide commands and operating parameters, such as by way of a keyboard, keypad and/or a user pointing device such as a mouse, touch screen, trackball, light pen, or finger pad. The user input device 72 may include a voice recognition device in lieu of or in addition to a keyboard. The input device 72 may be connected to the ultrasound unit 40 by a cable 76, an infrared link, a radio link (such as Bluetooth), or the equivalent, each of which are commercially available. In an embodiment including a cable 76 between the input device 72 and the image display unit 70 or ultrasound unit 40, a standard connector 108, such as an Ethernet, Firewire or USB connector, may be included to permit easy connection to and disconnection from the input device 72.

A display monitor 73 may not be present as part of ultrasound unit 40. Any of many choices, sizes, and styles of a display 73 may be connected to ultrasound unit 40. For example, the external display monitor 73 may be a cathode ray tube, a liquid crystal display, a plasma display screen, “heads up” video goggles, a video projector, or any other graphical display device that may become available. The display monitor 73 may be large and may be located conveniently out of the way, such as a plasma screen hung from the ceiling or on a wall. The display monitor 73 may be positioned for better viewing by the physician. There may be more than one display 73, and a display may be positioned remotely, such as in another room or in a distant facility. The display 73 may be connected to the ultrasound unit 40 by a cable, an infrared link, a radio link (such as Bluetooth), or any equivalent wireless technology.

In an embodiment, the display monitor 73 and/or the user input device 72 may be embodied by a computer terminal, workstation, or personal computer such as a laptop computer. Such an embodiment can be configured to display the graphical output from the image rendering circuits 83 and to pass user inputs via the interactive control 80 to the ultrasound unit 40. Alternatively, in an embodiment in which the display monitor 73 and user input device 72 are provided by a computer system, the computer system may operate software enabling it to perform additional image processing on the data received from the ultrasound unit 40.

The ultrasound unit 40 may be powered by an external power source 59, as was previously discussed with respect to FIGS. 1A and 1B. The power source 59 of FIG. 9 may be a separate external power supply with provision to isolate the ultrasound unit 40 from the patient. The power source 59 may be a power source, such as batteries, contained within the ultrasound unit 40. Alternatively, the power source 59 may simply be conductors in a data cable supplying power from input unit 72 or display unit 73.

Because of the priority of safety for a patient, a wired connection between the ultrasound unit 40 and the user input device 72 may need isolation circuitry to prevent potentially harmful leakage current from flowing from the input device 72 through the ultrasound unit 40 to the patient. This isolation may be provided by the isolation circuits 44 within the ultrasound unit 40 shown in FIGS. 1A, 1B, 5, 7 and 9. Alternatively or in addition, separate isolation circuits may be provided between the ultrasound unit 40 and the user input device 72, such as an optical fiber data link or optical isolation module as previously described. For example, if the input device 72 is powered from a source which shares a common ground (or other low impedance path) with the patient, there could be unexpected potential differences between the input device 72 and the patient conducted through the ultrasound unit 40. Such a potential difference might become a source of harmful leakage currents. Such currents are limited by sufficiently high impedance, such as provided by isolation circuitry, such as an optical isolator. The isolator may also protect against unintended high voltages caused by failures in the input device 72, its power source, static electricity, power surges or lightening, as well as reduce electronic noise induced by stray electromagnetic radiation. The same reasoning applies to a wired connection between the display 73 and the ultrasound unit 40, and any connection to a network.

One way to achieve isolation for the input device 72 or display device 73 is to employ a wireless communication link between the input device 72 and the ultrasound unit 40 and between the input device 73 and the ultrasound unit 40. Any wireless communication link of sufficient bandwidth, such as those mentioned previously, may be used in this capacity.

The embodiment illustrated by FIG. 9 includes an optional connection 68 between the ultrasound unit and an ECG sensor, such as an ECG catheter 64, and an optional connection 62 between the ultrasound unit 40 and an external ECG unit 60. In addition or alternatively, an embodiment may employ an ECG sensor integrated in the catheter 20 with a connection to the ultrasound unit 40 through the ultrasound data cable 28. The foregoing comments in the discussion of FIG. 6 about ECG signals also apply to an embodiment as illustrated by FIG. 9. The interface 62 may be any wired or wireless communication interface discussed previously. In an embodiment including a wired interface 62, a standard connector 105 (e.g., any of the example standard connectors listed herein with respect to other connectors in the ultrasound unit 40) may be provided in the ultrasound unit 40 to enable easy connection to and disconnection from the interface 62.

As illustrated in FIGS. 6 and 9, various embodiments of the ultrasound unit 40 can be housed within a housing 100 providing environmental and electrical isolation for the circuitry (e.g., circuit boards and integrated circuits) of the ultrasound unit 40. This housing 100 can be fabricated from nonconductive material, such as plastics, to minimize stray currents due to induction. The housing may also be fabricated from materials that can withstand high temperatures and/or gamma radiation so that the housing can be sterilized by heat or exposure to gamma rays.

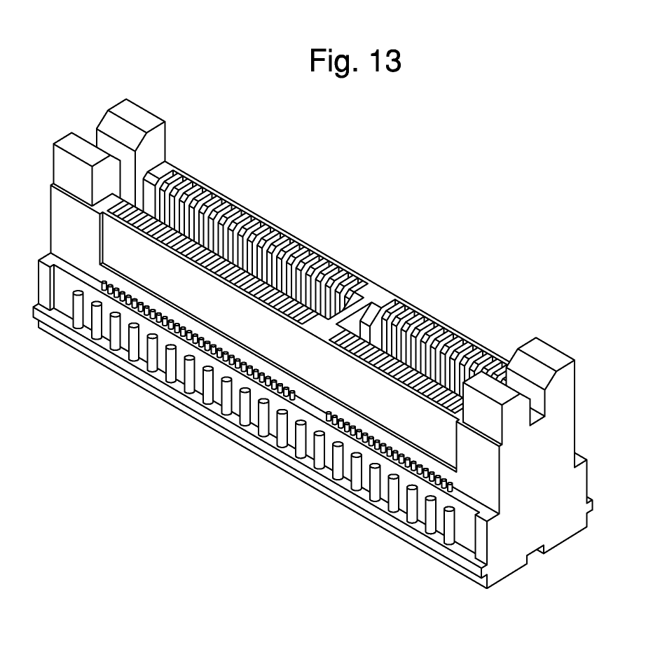

Referring to FIGS. 6 and 9, the housing 100 can include electrical connectors 102, 103, 104, 105, 106, 107, 108, 109 to permit the ultrasound unit 40 to be quickly connected to sensors, power, a user interface and displays, and to maintain a fluid or sterile boundary between the exterior and interior of the housing. For example, one or more multi-contact ultrasound catheter connectors 102 may be provided to enable a reliable electrical connection between an ultrasound phased array catheter 20 cable 28 plug and the ultrasound unit 40 while maintaining an environmental seal. An example of a suitable ultrasound catheter connector 102 is a card edge connector, such as the 0.762 mm Pitch Hi-SpecGS™ Memory Expansion Card Edge Connector manufactured by Molex Corporation (www.molex.com) illustrated in FIG. 13. Such an ultrasound catheter connector 102 can be provided on the interior of the housing 100. Similarly, one or more multi-contact ultrasound connectors 103 may be provided to enable a reliable electrical connection between an external ultrasound phased array 30 cable 38 plug and the ultrasound unit 40 while maintaining an environmental seal. The ultrasound connectors 102, 103 can be directly connected to the isolation circuits 44, with an electrical lead from a temperature sensor 26 connected to the thermal monitor circuit 42. A card connector (which is a flat plug) on the cable 28, 38 can be used to pass through a sterile plastic barrier to establish an electrical connection with the connector 2 102, 103. In this manner, a sterile plastic barrier can be used to serve as a boundary between sterile and non-sterile environments, and may be disposable to allow re-use of one or more of the various components. As described above, the plastic barrier may comprise, for example, a plastic sleeve or bag that encloses the housing 100 so the ultrasound unit 40 can be positioned near or on the patient and within a sterile boundary.

Additionally, one or more ECG sensor electrical connectors 104 may be provided in the housing 100 to permit electrically connecting ECG sensor connectors 68 to the ultrasound unit 40 without compromising the environmental seal of the housing 100. On the inside of the housing 100, the ECG sensor electrical connectors 104 may be electrically coupled to the isolation circuit 44 or pass through to an external ECG unit 60 via an ECG unit connector 105. In an embodiment, the housing 100 includes sixty-four or more contact edge connector type ports 104 for connecting sixty-four individual ECG probe elements.

Similarly, a power connector 106 may be provided in the housing 100 for accepting a connector to an external power source 59. Alternatively or in addition, an internal power source, such as an optional battery 110 can be included in the housing 100.

As mentioned above, an output connector 107 may be provided for connecting the ultrasound unit 40 to an external computer or display unit 73. For example, the output connector 107 may be a video connector (e.g., a CGI, VGA, EVGA or composite video connector) or a standard two-direction serial connector such as USB, FireWire or fiber optic connector. Similarly, an input connector 108 may be provided in the housing 100 to connect to user interface devices, such as a keyboard or computer 72. Likewise, a network cable connector 109 may be provided for directly connecting the ultrasound unit 40 to a network.

An advantage to all the embodiments described above is that they enable the use of short data cables between the ultrasound transducers and the beam former and ultrasound system. Shorter cables significantly reduce the electronic noise that is induced in cables from electromagnetic radiation and magnetic fields within the examination room. By positioning the analog-to-digital conversion electronics (i.e., the electronics which receive ultrasound signals from the transducers and convert the information into digital format) on or next to the patient, the information within the ultrasound signals is converted to noise-resistant digital format with the least amount of induced noise. By reducing the noise in the system, greater sensitivity and better ultrasound image resolution can be obtained. This advantage is further enhanced by embodiments employing a battery powered beam former/isolation ultrasound unit since such embodiments eliminate power cables and connections to the commercial power grid. This advantage is further enhanced by embodiments which employ wireless data links for communicating ultrasound image data to external displays and for receiving control commands from external user interface devices, thereby eliminating data cables connecting to the ultrasound unit.

Another advantage to all the embodiments described above is that they allow a compact ultrasound unit 40 to be enclosed in a hand-portable housing 100 that can be conveniently located near the patient without occupying a lot of space in the examination or operating room. This arrangement obviates the need for a cumbersome cable extending from the vicinity of the patient out of the sterile field. This arrangement may entirely obviate the need for an ultrasound cart. At most, one or two lighter, more flexible and manageable cable(s), if any, may be used to extend from the ultrasound unit 40 to the display 73 and to the ultrasound unit 40 from the user input device 72.

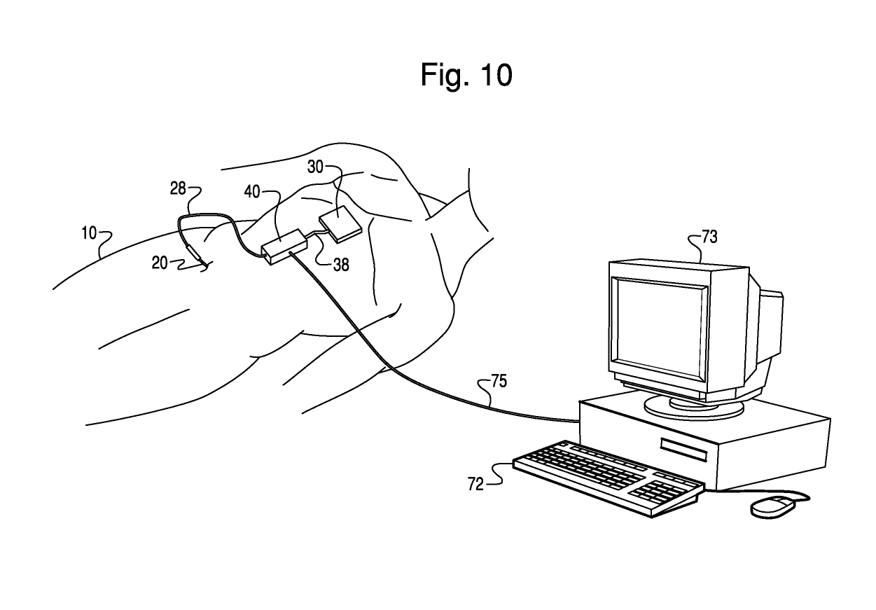

In particular, the small and portable nature of the ultrasound unit 40 and housing 100 enables placing the unit on the patient 10 so that a short cable 38 can be used to connect to the external ultrasound transducer assembly 30, as illustrated in FIG. 10. Such a short cable 38 can be configured so that it presents little or nor pressure or twisting force on the transducer assembly 30. So configured, the transducer assembly 30 can be positioned on the patient 10 and maintain a sonic connection with the patient's body so that ultrasound imaging can take place without a clinician repositioning the transducer assembly 30. This permits an ultrasound imaging procedure to include both a catheter transducer assembly 20 and an external transducer assembly 30 operating simultaneously without the need for additional clinicians.

While the various embodiments are illustrated and described as having a single catheter transducer assembly 20 and a single external transducer assembly 30, the ultrasound unit 40 may be configured to connect and operate with any number of catheter transducer assemblies and any number of external transducer assemblies by providing more connectors in the housing 100 and expanding the capacity of the internal circuitry. The capacity of the ultrasound unit 40 to power and manage multiple catheter and external transducer assemblies may be provided by replicating one or more of the modules described herein or increasing the capacity of such modules. Further, methods for concurrently operating a catheter transducer assembly and an external transducer assembly described herein may be expanded and used to operating multiple catheter and external transducer assemblies simultaneously. By operating multiple catheter and external transducer assemblies simultaneously, more and better ultrasound views may be obtained of a patient in order to better diagnose conditions and inspect an organ.

The various embodiments may be used according to the following method, wherein the steps can be performed in an order other than that described below. At least some of the steps may be performed contemporaneously. Some of the steps are optional.

An ultrasound unit 40, such as shown in FIG. 9, may be sterilized or placed inside an externally sterile enclosure. The ultrasound unit 40 may be positioned on a patient, such as illustrated in FIG. 10. The ultrasound unit 40 may be connected to an external power source or an internal power source (as in the embodiment shown in FIG. 10).

A user input device 72 may be supplied and connected to the ultrasound unit 40. A display monitor 73 may be supplied and connected to the ultrasound unit 40. As shown in the embodiment in FIG. 10, the user input device 72 and display monitor 73 may be a personal computer (such as a laptop) and the connections may be provided by a single two-way data cable 75. A data interface 92 may be established between the unit 40 and a clinic or hospital infrastructure 90.

A sterile, relatively short, catheter transducer cable 28 may be connected between the ultrasound unit 40 and a catheter transducer assembly 20. The catheter transducer assembly 20 may be introduced into a body, such as by percutaneous cannulation via the femoral artery as illustrated in FIG. 10, and positioned at a desired location and orientation to view the organ to be imaged, such as the heart. The catheter may be guided during cannulation by use of fluoroscopy.

Similarly, a short external transducer cable 32 may be connected between the ultrasound unit 40 and an external transducer assembly 30. The external transducer assembly 30 may be positioned on the patient 10 so as to be able to view the organ to be imaged, such as the heart, as illustrated in FIG. 10.

The unit 40 may be initialized and configured by an operator, such as interactively through an input device 72 and video monitor 73. The configuration may include setting of operational parameters of the ultrasound unit 40, such as ultrasound frequency, mode of operation, mode of image processing, characteristics of the transducer arrays 22, 32, anatomical position of the arrays 22, 32, details about the patient, and so forth. The operating parameters may be changed during operation of the ultrasound system. For example, the frequency may be changed to increase or decrease the depth of penetration of the ultrasound energy. As another example, the mode of display may be changed from B-mode (which may help the operator position the array 22 with respect to anatomy) to Doppler mode (e.g., color Doppler) to observe and measure velocity of motion of blood. Image data may be stored in a computer or transmitted via a connected network to a database server.

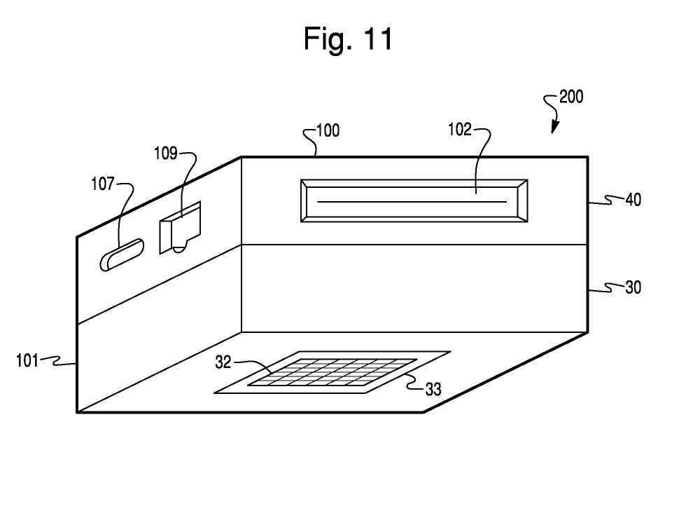

The small and portable nature of the ultrasound unit 40 and housing 100 also enables embodiments in which the external transducer assembly 30 is integrated with the ultrasound unit 40 as a single package 200, as illustrated in FIG. 11. Combining the external transducer assembly 30 and ultrasound unit 40 as a package reduces the number of components used in a procedure and eliminates a data cable 38 and the difficulties such a cable can present in a procedure. In an embodiment, the external transducer assembly 30 and ultrasound unit 40 are included within the same housing 100 which has an optical window 33 positioned on a face through which ultrasound can be transmitted and received by the transducer array 32. In such an embodiment, electronic connections between the transducer array 32 and the ultrasound unit 40 data processing circuitry 46, 48, 51 and electrical isolation circuitry 44 are included within the housing 100. In an alternative embodiment, the external transducer assembly 30 is contained within a housing 101 that electrically and, optionally, mechanically connects to the ultrasound unit 40 housing 100 to enable assembling a combined package 200. In such an embodiment, a mechanical coupling, such as a snap fitting, bayonet fitting or external strap, may be provided to securely hold the two housings 100, 101 together. Also, an electrical connector assembly (e.g., plug and connector) may be provided on interface surfaces of the two housings 100, 101, to electrically connect the external transducer assembly 30 to the ultrasound unit 40.

In use, a combined ultrasound unit/external transducer array package 200 can be positioned on a patient at a location to enable viewing an organ to be imaged, such as the heart. A catheter transducer assembly 20 may then be connected by inserting the catheter electrical connection plug into a catheter cable connector 102 and external display and control units may be connected via connector 107. Additionally, an external network may be connected, such as by an Ethernet cable plugged into a connector 109. With the system components so connected, imaging may then proceed as described above.

The various embodiments may employ one or more of a variety of methods to ensure that the ultrasound signals from the two (or more) transducers do not interfere with each other and can be individually detected and processed. The following paragraphs describe some example methods that can be used to deconflict ultrasound signals from multiple transducers. These descriptions are not intended to be exhaustive, and alternative methods and modifications of the following methods may also be used in various embodiments.

A simple method of avoiding interference is to alternately emit pulses from one transducer while the other transducer pauses. In this method, the ultrasound unit 40 first pulses one transducer, waits sufficient time for all echoes to be received by the emitting transducer array, and then pulses the other transducer(s). In this method, a single pulse may be emitted alternately by the transducers, or a number of pulses may be emitted first by one transducer and then by the other. Thus, in this alternating pulse method, the transducers essentially take turns emitting pulses. The silent transducer (or transducers when more than two transducers are used simultaneously) may be inactive or may continue to receive ultrasound, such as sound passing through tissue from the emitting transducer that may be processed for biphasic imaging as described herein.

Another method for deconflicting ultrasound signals emitted by multiple transducers employs different ultrasound frequencies used by each transducer. In this method, the different transducers emit ultrasound at different frequencies. Thus, ultrasound arriving at each transducer array can be differentiated based upon its frequency. Ultrasound signals can then be recognized by a digital signal processor within the ultrasound unit 40 so that received echoes of ultrasound emitted by a transducer can be distinguished from ultrasound transmitting through tissue from another transducer. Ultrasound passes through and interacts with tissue differently depending upon its frequency. For example, lower frequency ultrasound will penetrate deeper into tissues, enabling imaging of more distant structures, but is unable to image fine details in the structures. Higher frequency ultrasound can image fine structure details, but is rapidly attenuated in tissue. Thus, it may be desirable to operate the two (or more) transducers at different frequencies to take advantage of different imaging depths and sensitivities of different frequencies. For example, the external transducer may be operated at lower frequencies since it likely will be positioned further away from the organ to be imaged than a catheter-based transducer positioned within or adjacent to the organ. In this operating option, the external transducer may be used to obtain an overall image of the organ while detail images of selected portions of the organ are obtained using the catheter-based transducer positioned within or adjacent the organ.

Another method for deconflicting ultrasound signals emitted by multiple transducers employs identifying codes included within each ultrasound pulse to identify the respective transducer source. As a simple example, one transducer may emit a single pulse while another transducer emits a short train of two or more pulses. In this example embodiment, ultrasound may then be distinguished as to its source transducer by noting the number pulses within the sound. As another example, each transducer's pulse may include have a different pulse width that the ultrasound system can recognize. As a further example, the pulse emitted by each transducer may be exhibit a recognizable pulse shape or characteristic.

Another method for deconflicting ultrasound signals emitted by multiple transducers employs signal processing techniques to distinguish sound based upon characteristics such as amplitude, delay and direction. For example, ultrasound arriving from another transducer may have a much higher amplitude (i.e., louder) than ultrasound from that is echoing off of tissue. Thus, in this example, signal processing may be used to simply ignore ultrasound above a threshold amplitude, or to process the higher amplitude signals for biphasic imaging while processing lower amplitude signals for conventional B-mode, M-mode or Doppler imaging. Also, ultrasound received after the time of arrival of echoes at the maximum imaging depth (i.e., sound arriving after Tₘₐₓ=maximum imaging depth÷speed of sound in tissue) may be assumed to be from another transducer (or reflections of ultrasound emitted by another transducer). Thus, in this example, signal processing may be used to simply ignore ultrasound signals arriving after echoes at the maximum imaging depth. This technique may be used in combination with the above described method of alternating pulses among the transducers to maximize the pulse rate of all transducers (i.e., the delay between pulse in each transducer is approximately (ID_{transducer1}+ID_{transducer2})/Sₜᵢₛₛᵤₑ, where ID is imaging depth and Sₜᵢₛₛᵤₑ is the speed of sound in tissue).

Two or more of these example techniques may be combined to further deconflict ultrasound. For example, the different transducers may emit ultrasound at different frequencies and with imbedded codes, allowing ultrasound signals to be differentiated by the ultrasound unit 40 using both frequency and code recognition methods. As another example, the transducers may be alternately pulsed at different frequencies. This technique may allow long delayed echoes to be received, thereby allowing shorter intervals between pulses on each transducer.

Such techniques for preventing interference or distinguishing ultrasound data may be implemented within the ultrasound unit 40 by a variety of circuitry and software methods. For the alternately pulsing method, the ultrasound unit 40 can simply distinguish the ultrasound data based upon the time period in which it is received. For methods enabling simultaneous pulses, such as the frequency-distinguished and code-distinguished methods, the ultrasound may be digitized by a digital signal processor and then analyzed digitally to recognize the source of the ultrasound.

By enabling simultaneous imaging from two or more ultrasound transducers, the various embodiments enable a variety of imaging modes and procedures not available using conventional methods. For example, images from two or more transducer arrays may be used to image portions of an organ simultaneously that could not be imaged by one transducer at a time, such as the exterior portions of an organ that are beyond the imaging depth of an internally positioned sensor. As another example, the two or more transducers may be operated in different modes, such as B-Mode on one to image organ structures and color Doppler mode on another to measure the velocity and direction of movement of tissue and blood.

In embodiments that use more than one catheter transducer array and/or more than one external transducer, the aforementioned methods for deconflicting and coordinating multiple ultrasound transducers may be expanded to further enhance the effectiveness of the ultrasound imager. With more transducers, there may be opportunities to configure the transducer signals so the sensors work synergistically to better examine an organ. For example, two internal transducer arrays may be positioned to view the same region and operated at different frequencies and/or power level to image tissue at different depths and at different frequencies. Lower frequencies penetrate deeper enabling imaging of more distant structures but at lower resolution, so such a dual imaging application may allow higher resolution of close-in structures while simultaneously viewing more distant structures. As another example, two internal transducer arrays may be positioned and oriented to view different portions of the organ simultaneously, such as one positioned to image the ventricle while the other is positioned to image the atrium so that the shapes and interactive functioning of these connected regions of the heart can be assessed. Similar use of two external ultrasound imaging transducers may be made. As another example, one or more transducer arrays may be positioned and operated to measure blood flow velocity (i.e., operated in Doppler mode) while another one or more transducer arrays are positioned to image the same region in B-mode to measure the area of flow, thereby providing an accurate measure of instantaneous flow volume and other hydrodynamic properties. As another example, two or more transducer arrays may be positioned in different locations but oriented to view the same region so that stereo (or higher order) images can be obtained for generation of instantaneous 3-D and 4-D images. As yet another example, one transducer may be operated and used to provide feedback for other transducers for real time searching and monitoring. A further example described in more detail below positions two transducers to view the same ventricle from near orthogonal orientations in order to provide orthogonal ultrasound imaging of the ventricle.

In an embodiment, the ultrasound unit 40 is further configured, such as with software programmed into the control unit, to sequentially and adaptively controlled internal and external transducers working separately and simultaneously. By controlling multiple transducer sensors with such a smart hardware control circuit in the ultrasound unit 40 (or from a remote computer system), better use may be made of the capabilities of simultaneous ultrasound imaging. Further, such adaptive capability can enable the working mode to be versatile and capable of optimizing ultrasound parameters according to the particular application and situation. For example, the ultrasound unit 40 may monitor the degree of interference or artifacts created in each ultrasound image from signals of the other transducer array(s), and automatically adjust deconflicting methods and parameters (e.g., pulse delay or off-set, transducer duty cycles, pulse frequency or encoding method) to improve the obtained images. As another example, the ultrasound unit 40 may use the pulses generated by one transducer to calibrate or otherwise adjust signal reception and processing parameters applied to the other transducer(s).

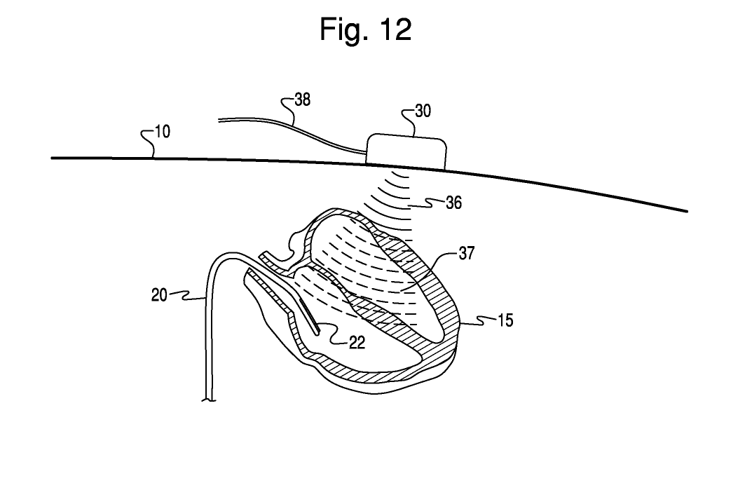

As mentioned above, an operational mode enabled by the various embodiments involves received and processing ultrasound emitted by one transducer that is received by another transducer. As illustrated in FIG. 12, such biphasic imaging takes advantage of the fact that sound travels at different speeds through different tissues and is scattered in different directions by tissue and bone, is not simply just reflected. In this operational mode, instead of ignoring signals from the other transducer, transmitted ultrasound 36 emitted by one transducer 30 is received by the other transducer array 22, with the signals processed to extract imaging information. Ultrasound from the transducer 30 will be slowed, scattered and/or redirected by tissue, such as a ventricle wall 15. Sound waves 37 that have passed through the tissue structure (e.g., ventricle wall 15) will be altered in direction, intensity and arrival time. Thus, the received ultrasound will arrive at the receiving transducer array 22 from different directions and at different times including information regarding the tissues between the two transducers. By knowing the distance and direction to the emitting transducer 30 (which may be determined from a first arriving and highest amplitude pulse or by means of other transducer locating techniques such as fluoroscopy), information about the intervening tissue may be obtained by measuring the angle, amplitude and delay of redirected ultrasound received on the receiving transducer array 22. In a sense, the emitting transducer 30 may serve as an illuminator for an imaging transducer array 22. Information regarding tissues obtained by such biphasic imaging may then be combined with image data obtained via conventional B-Mode, M-Mode or color Doppler direct imaging by either or both of the transducer arrays 22, 32. In an embodiment in which the transducer arrays 22, 32 are alternately pulsed, both transducer arrays may collect biphasic image data during the interval when the other array is emitting ultrasound. Biphasic imaging may also be accomplished when the transducers emit ultrasound at different frequencies (i.e., frequency deconfliction) or include coding (code-based deconfliction) that can enable the ultrasound system to distinguish monophasic (i.e., direct echoes) from biphasic ultrasound signals and apply the appropriate imaging process to the sound signals accordingly. Thus, the deconflicting embodiments may allow obtaining both conventional and biphasic ultrasound imaging from both transducers simultaneously.

A further operational mode enabled by the various embodiments involves three-dimensional ultrasound images that are obtained using an external three-dimensional imaging matrix transducer as are known in the ultrasound arts, which can be used in combination with high resolution two-dimensional slice images obtained from a catheter-based ultrasound imaging transducer. Three-dimensional imaging transducers include a matrix (i.e., an array) of transducers (such as several rows of linear transducer arrays). An array of transducers can be operated as a phased array to direct the ultrasound beam in two dimensions (e.g., up/down and left/right) and discriminate echoes arriving from two dimensions. Coupled to a beam former configured to generate and process such phased array signals, such a transducer can provide an instantaneous three-dimensional image of the examined volume of a patient. Such images may be very useful for imaging an entire organ, observing motion of the organ (e.g., the heart) as a whole, and identifying portions of the organ requiring close examination. However, as noted above, ultrasound images obtained through the skin of some organs, particularly the heart which is positioned behind the ribs, may have insufficient resolution for some diagnostic purposes. Due to venal diameter restrictions on catheters and physical limits on transducer size due to the physics of ultrasound waves, it is not possible to employ an effective three-dimensional ultrasound array within a percutaneous catheter. Nevertheless, percutaneous ultrasound imaging with two-dimensional phased array transducers can provide detailed images of narrow slices of the organ to provide high resolution imaging required for some diagnostic examinations. Thus, external three-dimensional ultrasound transducers can be used synergistically with two-dimensional percutaneous ultrasound transducer to provide three-dimensional images of organs with internal high resolution two-dimensional slice images of selected portions.

A further operational mode enabled by the various embodiments involves presenting an ultrasound image from one transducer (either external or percutaneous) as an overlay or adjacent window within the display so that the clinician can view both images simultaneously. Such picture-in-picture presentation is an operational mode that may be selected in an embodiment in order to give the clinician flexibility in using the visual information provided by the two imaging perspectives. This picture-in-picture format may be used to present the internal high resolution 2-D slice images as a window or picture within a display of a three-dimensional view obtained from a three-dimensional external ultrasound imaging transducer.

A further operational mode is enabled by the various embodiments in which information from internal high resolution 2-D slice ultrasound images are used to provide the effect of a high resolution “zoom lens” within a display of a three dimensional view obtained from a three-dimensional external ultrasound imaging transducer. During real-time viewing, the clinician may zoom in to a portion of the 3-D image that is also imaged by the percutaneous ultrasound transducer. During off-line viewing (i.e., reviewing ultrasound image data stored in memory), this function may be extended to enable high resolution zoom magnification of those portions of the 3-D image for which internal high resolution 2-D slice ultrasound images were obtained and stored in memory. This operational mode or system tool should help clinicians to make use of the diagnostic information available in both the global 3-D and internal high resolution 2-D slice ultrasound images in an intuitive manner.

A further operational mode enabled by the various embodiments provides orthogonal ultrasound imaging in which two simultaneous near-orthogonal two-dimensional ultrasound images are obtained of an organ. Orthogonal imaging of the chambers of the heart can be very useful in diagnosing ventricular function and determining appropriate therapies, in particular bi-ventricular pacing. This is because a two-dimensional slice image of a ventricle reveals contraction only within the image plane (i.e., as a reduction in the imaged area of the ventricle). A diseased heart may contract properly along one dimension but not along other dimensions. In such cases, the ventricle may expand along the other dimensions so that a portion of the blood volume is not expelled and instead sloshes to weaker or non-contracting portions of the ventricle. Such conditions are easily detected by simultaneous ultrasound imaging of the ventricle along two (or more) near-orthogonal planes. Such imaging of a healthy heart will show contractions in both imaging planes, while a diseased heart will be revealed by unequal contractions in the two imaging planes. The various embodiments enable orthogonal ultrasound imaging from an external transducer and a percutaneous transducer, or from two percutaneous transducers. For example, the viewing orientation of the percutaneous transducer can be determined (such as by using fluoroscopy) and then the external transducer orientation can be adjusted (such as rotating the transducer) to provide an orthogonal viewing perspective.

Other imaging sensor technology may also be combined or “fused” with images from a multiple transducer and internal and external ultrasound imaging system to provide better or more accurate imaging. For example, location sensing systems can be used to locate the position of each transducer array within a frame of reference (e.g., a patient-centered or external frame of reference) in order to register the resulting images in 3-D space. By registering the ultrasound images (particularly 3-D and 4-D images as described above) in a frame of reference, the image information can be fused with other imaging technologies, such as fluoroscopy, to provide a multi-sensor image of an organ. Also, 3-D and 4-D images registered in a frame of reference may be used with image guided surgery systems to enable precision surgery on the imaged organ.

It is noted that internal ultrasound imaging and external ultrasound imaging techniques are different and will result in different image perspectives with different qualities. Such differences in image content and quality may need to be accounted for when fusing image information from the two types of ultrasound transducers. For example, external transducers may employ higher power levels and lower ultrasound frequencies, allowing them to image deeper, but the resulting resolution will be different from the resolution of an internal transducer image. Thus, a pixel in an external transducer ultrasound image may represent a larger volume of space than does a pixel from the internal transducer image. As another example, the difficulty of accurately locating a catheter tip within a patient's body means that there will likely be larger position errors associated with the internal transducer image information than may be the case for the external transducer image information. Thus, image fusion methods employed may need to account and correct for differences in pixel size and differences in positional accuracy inherent in the internal and external transducer image information.

An embodiment includes some or all of the components described herein as a packaged kit with previously sterilized contents. The contents may include, by way of non-limiting example, a catheter 20 bearing an ultrasound transducer array 22, one or more sterile cables, a sterile enclosure for the ultrasound unit 40, a battery for the ultrasound unit 40 (if it is battery powered), and instructions. The kit may further include a cable to connect the ultrasound transducer array 22 to the ultrasound unit 40, as well as the ultrasound transducer array 22. The kit may include a cable to connect the ultrasound unit 40 to the display unit 70, unless the connection between them is wireless. In lieu of just a sterile enclosure for the ultrasound unit 40, an embodiment of the kit may contain the ultrasound unit 40 itself, which has been previously sterilized. Any appropriate method may be used to sterilize the contents, such as gamma radiation, which is typically used to sterilize packaged kits in bulk. Some or all of the contents may be disposable or may be sterilizable for reuse.

While the present invention has been disclosed with reference to certain exemplary embodiments, numerous modifications, alterations, and changes to the described embodiments are possible without departing from the sphere and scope of the present invention, as defined in the appended claims. Accordingly, it is intended that the present invention not be limited to the described embodiments, but that it have the full scope defined by the language of the following claims, and equivalents thereof.

We claim:

1. An ultrasound system, comprising:
a catheter including
an ultrasound transducer array positioned near a distal end of the catheter, and
a temperature sensor positioned near the ultrasound transducer array;
an external transducer array; and
an integrated ultrasound unit electrically coupled to the catheter and the external transducer array, the ultrasound unit comprising:
a housing;
an electrical isolation circuit positioned within the housing
an ultrasound signal generator positioned within the housing and coupled to the electrical isolation circuit;
a beam-forming circuit positioned within the housing and coupled to the electrical isolation circuit; and
a communication interface circuit positioned within the housing and electronically coupled to the beam forming circuit, the communication interface circuit configured to transmit data to an image display unit.

2. The ultrasound system according to claim 1, further comprising a control unit positioned within the housing and electronically coupled to the ultrasound signal generator and beam-forming circuit.

3. The ultrasound system according to claim 1, further comprising a thermal monitor circuit positioned within the housing, the thermal monitor circuit configured to receive a temperature input signal from the temperature sensor and discontinue transmission of ultrasound signals from the signal generator through the electrical isolation circuit when the temperature input signal indicates a sensed temperature exceeding a threshold.

4. The ultrasound system according to claim 1, further comprising a connector positioned on the housing and electrically coupled to the communication interface circuit, the connector configured to receive a data transmission cable for connecting to the image display unit.

5. The ultrasound system according to claim 1, further comprising a wireless data link circuit positioned within the housing and coupled to the communication interface circuit, the wireless data link circuit configured to transmit data to the image display unit by a wireless data link signal.

6. The ultrasound system according to claim 1, wherein the integrated ultrasound unit is configured to enable simultaneous imaging by both the catheter ultrasound transducer array and the external transducer array.

7. The ultrasound system according to claim 6, wherein the integrated ultrasound unit is configured to transmit ultrasound pulse signals to one of the catheter ultrasound transducer array and the external transducer array at a time.

8. The ultrasound system according to claim 6, wherein the integrated ultrasound unit is configured to transmit ultrasound pulse signals to the catheter ultrasound transducer array at a first frequency and to the external transducer array at a second frequency different from the first frequency.

9. The ultrasound system according to claim 8, wherein the integrated ultrasound unit is configured to distinguish received ultrasound signals based upon the frequency of ultrasound received by one of the catheter ultrasound transducer array and the external transducer array.

10. The ultrasound system according to claim 6, wherein the integrated ultrasound unit is configured to transmit ultrasound pulse signals to the catheter ultrasound transducer array including a first code and to the external transducer array including a second code different from the first code.

11. The ultrasound system according to claim 10, wherein the integrated ultrasound unit is configured to distinguish received ultrasound signals based upon the first and second codes included in ultrasound received by one of the catheter ultrasound transducer array and the external transducer array.

12. The ultrasound system according to claim 1, wherein the external transducer array is positioned within the housing.

13. A portable integrated ultrasound unit, comprising:
a housing;
an electrical isolation circuit positioned within the housing;
an ultrasound signal generator positioned within the housing and electrically coupled to the electrical isolation circuit and configured to generate ultrasound signals for two transducer arrays; and
a beam-forming circuit positioned within the housing and electrically coupled to the electrical isolation circuit, the beam-forming circuit configured to receive ultrasound signals from two transducer arrays via the electrical isolation circuit and output ultrasound data.

14. The portable integrated ultrasound unit according to claim 13, further comprising a control unit positioned within the housing and electronically coupled to the ultrasound signal generator and beam-forming circuit.

15. The portable integrated ultrasound unit according to claim 13, further comprising a thermal monitor circuit positioned within the housing, the thermal monitor circuit configured to receive a temperature input signal from a temperature sensor and discontinue transmission of ultrasound signals from the signal generator through the electrical isolation circuit when the temperature input signal indicates a sensed temperature exceeding a threshold.

16. The portable integrated ultrasound unit according to claim 13, further comprising a communication interface circuit positioned within the housing and coupled to the beam former circuit, the communication interface circuit configured to transmit data to an external display.

17. The portable integrated ultrasound unit according to claim 16, further comprising:
a first connector positioned on the housing and electrically coupled to the isolation circuit, the first connector configured to receive electrical leads from a catheter-based ultrasound transducer array; and
a second connector positioned on the housing and electrically coupled to the beam forming circuit, the second connector configured to receive electrical leads from an external ultrasound transducer array.

18. The portable integrated ultrasound unit according to claim 17, further comprising a wireless data link circuit positioned within the housing and coupled to the communication interface circuit, the wireless data link circuit configured to transmit data to an external display by a wireless data link signal.

19. The portable integrated ultrasound unit according to claim 13, wherein the ultrasound signal generator is configured to transmit ultrasound pulse signals to one of the catheter ultrasound transducer array and the external transducer array at a time.

20. The portable integrated ultrasound unit according to claim 19, wherein the ultrasound signal generator is configured to transmit ultrasound pulse signals to the catheter ultrasound transducer array at a first frequency and to the external transducer array at a second frequency different from the first frequency.

21. The portable integrated ultrasound unit according to claim 19, wherein the portable integrated ultrasound unit is configured to distinguish received ultrasound signals based upon the frequency of ultrasound received by one of the catheter ultrasound transducer array and the external transducer array.

22. The portable integrated ultrasound unit according to claim 19, wherein the ultrasound signal generator is configured to transmit ultrasound pulse signals to the catheter ultrasound transducer array including a first code and to the external transducer array including a second code different from the first code.

23. The portable integrated ultrasound unit according to claim 22, wherein the portable integrated ultrasound unit is configured to distinguish received ultrasound signals based upon the first and second codes included in ultrasound received by one of the catheter ultrasound transducer array and the external transducer array.

24. The portable integrated ultrasound unit according to claim 13, further comprising an external transducer array positioned within the housing.

25. A method for imaging an organ within a body, comprising:
positioning a catheter within the body near or within the organ, the catheter including a first ultrasound transducer array positioned near a distal end of the catheter;
positioning a second ultrasound transducer array on the body;
providing signals to the first and second ultrasound transducer arrays to enable simultaneous ultrasound imaging of the organ;
receiving signals from the first and second ultrasound transducer arrays; and
generating ultrasound images of the organ based upon the received signals.

26. The method of imaging an organ in a body according to claim 25, further comprising transmitting ultrasound pulses from one of the first ultrasound transducer array and the second ultrasound transducer array at a time.

27. The method of imaging an organ in a body according to claim 25, further comprising transmitting ultrasound pulses from the first ultrasound transducer array at a first frequency and transmitting ultrasound pulses from the second transducer array at a second frequency different from the first frequency.

28. The method of imaging an organ in a body according to claim 27, further comprising distinguishing received ultrasound signals based upon the frequency of ultrasound received by one of the first and second ultrasound transducer arrays.

29. The method of imaging an organ in a body according to claim 25, further comprising transmitting ultrasound pulses from the first ultrasound transducer array including a first code and transmitting ultrasound pulses from the second transducer array including a second code different from the first code.

30. The method of imaging an organ in a body according to claim 29, further comprising distinguishing received ultrasound signals based upon the first and second codes included in ultrasound received by one of the first and second ultrasound transducer arrays.
